(19) United States
(12) Patent Application Publication
(10) Pub. No.: US 2018/0362624 A1
(43) Pub. Date: Dec. 20, 2018

(54) IG1 AND THE THERAPEUTIC USE THEREOF

(71) Applicant: UNIVERSITE FRANCOIS RABELAIS DE TOURS, Tours Cedex 1 (FR)

(72) Inventors: Valerie GOUILLEUX-GRUART, Tours (FR); Herve WATIER, Ballan-Mire (FR)

(21) Appl. No.: 15/748,415

(22) PCT Filed: Jul. 28, 2016
(86) PCT No.: PCT/FR2016/051973

§ 371 Date: Jan. 29, 2018

(30) Foreign Application Priority Data
Aug. 4, 2015 (FR) 1557535

(51) Int. Cl. C07K 16/18; A61K 39/395

(52) U.S. Cl. CPC C07K 16/18; A61K 39/39533; A61K 39/39591; A61K 2039/505; C07K 2317/526; C07K 2317/94; C07K 2317/522

(57) ABSTRACT

In the field of monoclonal antibodies for therapeutic use, in particular IgG1s for therapeutic use, there is disclosed a method for increasing the binding affinity of an IgG1 vis-à-vis the FcRn receptor, and/or increasing the stability of the complex formed by such IgG1 and FcRn. A related pharmaceutical composition is also disclosed.

The present invention relates to the field of monoclonal antibodies for therapeutic use, in particular the IgG1s.

Monoclonal antibodies currently represent a therapeutic alternative of prime importance in the treatment of an extremely wide variety of medical conditions. In many cases, the ability of the antibodies to specifically recognize an antigen by means of their Fab fragment and to recruit immunity effectors by means of their Fc fragment makes it possible to treat these diseases with a favourable risk-benefit balance.

The effectiveness of the treatments with therapeutic monoclonal antibodies is influenced by the dose administered and the frequency of administration of the antibody. As for any treatment, maintaining the effectiveness of the treatment combined with reducing the dose of therapeutic antibody administered or the frequency of administration is highly desirable, both from the economic point of view and for the comfort of the patient.

The dose administered and the frequency of administration depend on numerous factors, including in particular the half-life of the therapeutic antibody in the body of the patient treated. An increase in the half-life of the antibody in the body of the patient thus makes it possible to reduce the single dose administered, and also to increase the interval between two administrations.

The IgG1s (Immunoglobulins G of subclass 1) are constituted by an assembly of two dimers, each constituted by a heavy chain γ1 and a light chain assembled by means of a disulphide bridge. These two dimers are assembled together by means of two disulphide bridges between the heavy chains.

Each of the heavy chains and light chains is constituted by a constant region called CH in the case of the heavy chain (containing three domains CH1, CH2 and CH3) and CL in the case of the light chain (containing a single domain), and by a variable region called VH in the case of the heavy chain and VL in the case of the light chain respectively. The assembly of the chains which compose an antibody defines a three-dimensional structure constituted by three arms of almost equal size (each comprising 4 domains). These arms are joined by a hinge, and are divided between two Fab (antigen binding) arms and one Fc (crystallizable) arm. The Fc arm is constituted by the CH2 and CH3 domains of the two heavy chains. At its free end, each Fab arm is constituted by variable domains of light and heavy chains, each linked to the CL and CH1 domains respectively, the latter ensuring the link with the hinge.

Generally, the variable region is involved in the recognition of an epitope and the Fc fragment constitutes the support of the biological properties of the immunoglobulin, in particular its ability to be recognized by the cellular immunity effectors (such as the effector cells expressing Fcγ receptors), to activate the complement system and to bind to the FcRn (Neonatal Fc receptor).

It is now well known that the half-life of an antibody in the body of a patient is closely linked to its affinity for the FcRn protein, (Roopenian et al., 2007. FcRn: the neonatal Fc receptor comes of age. Nat. Rev. Immunol. 7: 715-725). In fact, the FcRn protects the antibodies from degradation, ensuring them a long half-life. This phenomenon is known as antibody recycling. It also ensures their distribution in the body by transporting them from one cellular compartment to another. These two phenomena involve the penetration of the IgGs into the cells by pinocytosis or endocytosis, then the recognition of the Fc fragment of the IgGs by the FcRn protein present in the endosomes. The IgGs bind specifically to the FcRn protein due to the acid pH prevailing within the endosomes, thus preserving the lysosomial catabolism and allowing their transport from one pole of the cell to the other in the case of cell transcytosis or towards the same cell pole in the event of their recycling (Oganesyan et al., 2014. Structural insights into neonatal Fc receptor-based recycling mechanisms. J Biol. Chem. 289: 7812-7824).

A major advance in understanding the interactions between the IgGs and the FcRn protein has been achieved through the determination of the structure of these proteins, in particular the determination of the IgG regions interacting with the FcRn. It has thus been demonstrated that the IgGs interact with this protein in a manner dependent on the pH at the level of the CH2-CH3 constant regions of the heavy chain (Shields et al., 2001. High resolution mapping of the binding site on human IgG1 for Fc gamma RI, Fc gamma RII, Fc gamma RIII, and FcRn and design of IgG1 variants with improved binding to the Fc gammaR. J. Biol. Chem. 276: 65-6604).

Recent studies have made it possible to identify amino acids of the peptide sequence of the Fc regions, at the level of the junction of the CH2-CH3 domains, in the constant region of the heavy chain the mutation of which can positively or negatively influence the binding affinity of an IgG1 for the FcRn protein. Some of these mutations make it possible to increase the binding affinity of the IgG1s for FcRn, and thus lead to an increase in the half-life of the IgG (Kuo et al., 2011. Neonatal Fc receptor and IgG-based therapeutics. Mabs 3: 422-430). However, none of the mutants envisaged has yet led to the approval and marketing of an antibody. For economic reasons and for reasons of comfort, the need still remains to find novel solutions for improving the half-life of the IgG1 antibodies.

A first aspect of the invention is thus to propose a method for improving the binding affinity and/or increasing the stability of the complex formed by said IgG1 and FcRn. The IgG1s obtained by this method thus have a better lifetime, making their therapeutic use easier and more effective, in particular while reducing the doses administered.

Another aspect of the invention is to propose the therapeutic use of IgG1 having a lifetime greater than that of most conventional therapeutic IgG1s.

Another aspect of the invention is to propose the therapeutic use of such IgG1s for treating certain classes of patients and/or certain specific diseases.

Another aspect of the invention is to propose pharmaceutical compositions containing such IgG1s.

The invention therefore relates to a method for improving the binding affinity of an IgG1 vis-à-vis the FcRn receptor, and/or increasing the stability of the complex formed by said IgG1 and FcRn, comprising a step in which the constant part of a heavy chain of an IgG1 of Glm3, Glm17 or Glm17,1 allotype is replaced with the constant part of a heavy chain of Glm3,1 allotype in order to obtain an IgG1 of Glm3,1 allotype,

the binding affinity of the IgG1 of Glm3,1 allotype vis-à-vis the FcRn receptor being greater than the binding affinity of the IgG1 of Glm3, Glm17 or Glm17,1 allotype vis-à-vis the FcRn receptor, and/or

the stability of the complex formed by the IgG1 of Glm3,1 allotype and the FcRn, being greater than the stability of the complex formed by the IgG1 of Glm3, Glm17 or Glm17,1 allotype and the FcRn receptor.

The present invention is based on the unexpected observation made by the Inventors that the IgG1s which differ only in their allotypes, i.e. in the combination of the variations of 3 amino acids located in the CH1 and CH3 parts, i.e. in the Fab and Fc parts of the IgG1, have different binding properties for the FcRn protein.

More particularly, the present invention is based on the observation that the IgG1s of Glm3,1 allotype bind to the FcRn receptor more effectively than the IgG1s of Glm3, Glm17 and Glm17,1 allotype.

The recycling and, consequently, the half-life duration of the IgG1s can thus be improved by replacing the constant region of the heavy chain of the Glm3, Glm17 and Glm17,1 IgG1s with the constant region of the heavy chain of Glm3,1 allotype.

Within the meaning of the present invention, the allotypes are determined by the natural variation, or polymorphism, in the peptide sequence of the constant region of the heavy chain of human IgG1s at positions 214, 356 and 358 (EU numbering). These positions correspond to the positions 120 of the CH1 region and 12 and 14 of the CH3 constant region according to IMGT numbering.

The allotype variations and their numbering are presented in Table 1.

TABLE 1
Allotypes of the IgG1s (G1m system).

| Nomenclature | Positions of the amino acids | | |
|---|---|---|---|
| EU | 214 | 356 | 358 |
| IMGT | 120 (CH1) | 12 (CH3) | 14 (CH3) |
| Exon numbering | 97 (CH1) | 16 (CH3) | 18 (CH3) |
| G1m3 | Arginine | Glutamate | Methionine |
| G1m3,1 | Arginine | Aspartate | Leucine |
| G1m17 | Lysine | Glutamate | Methionine |
| G1m17,1 | Lysine | Aspartate | Leucine |

In the present application, the position of the amino acids is indicated using EU numbering, as defined in Table 1.

In the invention, the expression “replacement of the constant region of the heavy chain of an IgG1 of Glm3, Glm17 or Glm17,1 allotype with that of an IgG1 of Glm3,1 allotype” denotes any modification, or transformation making it possible, starting from a Glm3, Glm17 or Glm17,1 IgG1 to arrive at an IgG1 of Glm3,1 allotype. In particular the point mutation of certain amino acids is possible, or it is also possible to replace certain domains (such as for example, to replace the CH1 and/or CH3 domains without modifying the CH2 domain).

In non-limitative manner, the replacement of the constant region of the heavy chain of an IgG1 of Glm3, Glm17 or Glm17,1 allotype with that of an IgG1 of Glm3,1 allotype can be carried out using conventional molecular biology tools, such as PCR, directed mutagenesis or cloning into appropriate vectors (such as for example the pFUSE-CH1g vector). Thus, it is possible to obtain in vitro different vectors allowing the expression of a complete IgG1 having a given allotype.

In the invention, the terms “constant part” and “constant region” are identical in meaning and can be used interchangeably.

In the invention, the terms “variable part” and “variable region” are identical in meaning and can be used interchangeably.

In an embodiment, the invention relates to a method as defined above, in which the binding affinity of the IgG1 of Glm3,1 allotype vis-à-vis the FcRn receptor, is at least 10% greater than the binding affinity of the IgG1 of Glm3, Glm17 or Glm17,1 allotype vis-à-vis the FcRn receptor.

In an embodiment, the invention relates to a method as defined above, in which the binding affinity of the IgG1 of Glm3,1 allotype vis-à-vis the FcRn receptor, is at least 20%, 30%, 40% or 50% greater than the binding affinity of the IgG1 of Glm3, Glm17 or Glm17,1 allotype vis-à-vis the FcRn receptor.

In an embodiment, the invention relates to a method as defined above, in which the stability of the complex formed by the IgG1 of Glm3,1 allotype and the FcRn, is at least 10%, preferably 40%, greater than the stability of the complex formed by the IgG1 of Glm3, Glm17 or Glm17,1 allotype and the FcRn receptor.

In an embodiment, the invention relates to a method as defined above, in which the stability of the complex formed by the IgG1 of Glm3,1 allotype and the FcRn, is at least 10%, 20%, 30%, 40% or 50% greater than the stability of the complex formed by the IgG1 of Glm3, Glm17 or Glm17,1 allotype and the FcRn receptor.

In a non-limitative manner, the binding affinity between an IgG1 and the FcRn, as well as the stability of the complex formed by an IgG1 and the FcRn, can be determined using conventional techniques making it possible to measure the interaction between a ligand and its receptor, such as for example SPR (surface plasmon resonance).

In non-limitative manner, the binding affinity of the IgG1s for FcRn is measured at acid pH by SPR using recombinant human FcRn covalently coupled by its primary amine functions to the surface of a CM5 dextran biosensor. The sensorgrams obtained on the measurement channel are assessed after subtraction of the response obtained on the control channel by means of software (such as Biaevaluation 4.2) with a bivalent calculation model (heterogeneous fit). This method makes it possible to assess the association and dissociation constants of the antibody with respect to the FcRn.

The use of Jurkat cells expressing the truncated FcRn of 33 amino acids in its C-terminal part, leading to the maintenance of the FcRn at the surface of cells, also makes it possible to assess the binding of antibody to the FcRn by flow cytometry measurement at acid pH. This analysis is carried out by competition using a fluorochrome-labelled reference antibody and allows the comparison of binding of different antibodies.

In a non-limitative manner, the stability of the IgG1/FcRn complex is assessed by SPR using recombinant human FcRn covalently coupled by its primary amine functions to the surface of a CM5 dextran biosensor. The stability is assessed based on sensorgrams during the dissociation phase.

In an embodiment, the invention relates to a method as defined above, in which the IgG1 of Glm3,1 allotype has a half-life duration greater than that of the IgG1 of Glm3, Glm17 or Glm17,1 allotype.

In an embodiment, the invention relates to a method as defined above, in which the IgG1 of Glm3,1 allotype, has a half-life duration at least 10%, 20%, 30%, 40% or 50% greater than that of the IgG1 of Glm3, Glm17 or Glm17,1 allotype.

In non-limitative manner, the half-life duration of the IgG1s is determined in a murine model expressing human FcRn. The different antibodies are injected into the mice. A blood sample is taken 2 hours, 6 hours, 1, 3, 7, 10, 14, 17 and 21 days after injection of the antibody in order to measure the blood concentrations which make it possible to calculate the half-life.

In an embodiment, the invention relates to a method as defined above in which the constant region of a heavy chain of an IgG1 of Glm3, Glm17 or Glm17,1 allotype is replaced with the constant region of a heavy chain of Glm3,1 allotype having at least 90% identity with SEQ ID NO: 1.

Table 2 shows a reference sequence for each of the 4 allotypes, Glm3,1, Glm3, Glm17 and Glm17,1. The natural variations determining the allotypes are underlined.

TABLE 2
Reference sequences of the allotypes.

| | Reference sequence of the constant |
|---|---|
| Allotype | region of the heavy chain of IgG1 |
| G1m3,1 | SEQ ID NO: 1 |
| | ASTKGPSVFPLAPSSKSTSGGTAALGCLVKDYFPEPV |
| | TVSWNSGALTSGVHTFPAVLQSSGLYSLSSVVTVPSS |
| | SLGTQTYICNVNHKPSNTKVDKRVEPKSCDKTHTCPP |
| | CPAPELLGGPSVFLFPPKPKDTLMISRTPEVTCVVVD |
| | VSHEDPEVKFNWYVDGVEVHNAKTKPREEQYNSTYRV |
| | VSVLTVLHQDWLNGKEYKCKVSNKALPAPIEKTISKA |
| | KGQPREPQVYTLPPSRDELTKNQVSLTCLVKGFYPSD |
| | IAVEWESNGQPENNYKTTPPVLDSDGSFFLYSKLTVD |
| | KSRWQQGNVFSCSVMHEALHNHYTQKSLSLSPGK |
| G1m3 | SEQ ID NO: 2 |
| | ASTKGPSVFPLAPSSKSTSGGTAALGCLVKDYFPEPV |
| | TVSWNSGALTSGVHTFPAVLQSSGLYSLSSVVTVPSS |
| | SLGTQTYICNVNHKPSNTKVDKRVEPKSCDKTHTCPP |
| | CPAPELLGGPSVFLFPPKPKDTLMISRTPEVTCVVVD |
| | VSHEDPEVKFNWYVDGVEVHNAKTKPREEQYNSTYRV |
| | VSVLTVLHQDWLNGKEYKCKVSNKALPAPIEKTISKA |
| | KGQPREPQVYTLPPSREEMTKNQVSLTCLVKGFYPSD |
| | IAVEWESNGQPENNYKTTPPVLDSDGSFFLYSKLTVD |
| | KSRWQQGNVFSCSVMHEALHNHYTQKSLSLSPGK |
| G1m17 | SEQ ID NO: 3 |
| | ASTKGPSVFPLAPSSKSTSGGTAALGCLVKDYFPEPV |
| | TVSWNSGALTSGVHTFPAVLQSSGLYSLSSVVTVPSS |
| | SLGTQTYICNVNHKPSNTKVDKKVEPKSCDKTHTCPP |
| | CPAPELLGGPSVFLFPPKPKDTLMISRTPEVTCVVVD |
| | VSHEDPEVKFNWYVDGVEVHNAKTKPREEQYNSTYRV |
| | VSVLTVLHQDWLNGKEYKCKVSNKALPAPIEKTISKA |
| | KGQPREPQVYTLPPSREEMTKNQVSLTCLVKGFYPSD |
| | IAVEWESNGQPENNYKTTPPVLDSDGSFFLYSKLTVD |
| | KSRWQQGNVFSCSVMHEALHNHYTQKSLSLSPGK |
| G1m17,1 | SEQ ID NO: 4 |
| | ASTKGPSVFPLAPSSKSTSGGTAALGCLVKDYFPEPV |
| | TVSWNSGALTSGVHTFPAVLQSSGLYSLSSVVTVPSS |
| | SLGTQTYICNVNHKPSNTKVDKKVEPKSCDKTHTCPP |
| | CPAPELLGGPSVFLFPPKPKDTLMISRTPEVTCVVVD |
| | VSHEDPEVKFNWYVDGVEVHNAKTKPREEQYNSTYRV |
| | VSVLTVLHQDWLNGKEYKCKVSNKALPAPIEKTISKA |
| | KGQPREPQVYTLPPSRDELTKNQVSLTCLVKGFYPSD |
| | IAVEWESNGQPENNYKTTPPVLDSDGSFFLYSKLTVD |
| | KSRWQQGNVFSCSVMHEALHNHYTQKSLSLSPGK |

In the invention, the term “IgG1 of Glm3,1 allotype” denotes an IgG1 having an arginine, an aspartate and a leucine at positions 214, 356 and 358 respectively. The constant region of its heavy chain is constituted by a sequence having at least 90%, in particular 91%, 92%, 93%, 94%, 95%, 96%, 97%, 98%, 99% or 100%, identity with the sequence SEQ ID NO: 1.

In the invention, the term “IgG1 of Glm3 allotype” (also denoted Glm3,-1) denotes an IgG1 having an arginine, a glutamate and a methionine at positions 214, 356 and 358 respectively. The constant region of the heavy chain is constituted by a sequence having at least 90%, in particular 91%, 92%, 93%, 94%, 95%, 96%, 97%, 98%, 99% or 100%, identity with the sequence SEQ ID NO: 2.

In the invention, the term “IgG1 of Glm17 allotype” (also denoted Glm17,-1) denotes an IgG1 having a lysine, a glutamate and a methionine at positions 214, 356 and 358 respectively. The constant region of its heavy chain is constituted by a sequence having at least 90%, in particular 91%, 92%, 93%, 94%, 95%, 96%, 97%, 98%, 99% or 100%, identity with the sequence SEQ ID NO: 3.

In the invention, the term “IgG1 of Glm17,1 allotype” denotes an IgG1 having a lysine, an aspartate and a leucine at positions 214, 356 and 358 respectively. The constant region of its heavy chain is constituted by a sequence having at least 90%, in particular 91%, 92%, 93%, 94%, 95%, 96%, 97%, 98%, 99% or 100%, identity with the sequence SEQ ID NO: 4.

Generally, the percentage identity between two sequences of nucleic acids or amino acids is determined by comparing these two optimally aligned sequences in which the sequence of nucleic acids or amino acids to be compared can comprise additions or deletions with respect to the reference sequence for an optimal alignment between these two sequences. The percentage identity is calculated by determining the number of identical positions for which the nucleotide or the amino acid residue is identical between the two sequences, by dividing this number of identical positions by the total number of positions in the comparison window and two sequences. The optimal alignment of the sequences for the comparison can be carried out by computer using known algorithms.

The constant region of the light chains of an IgG1 of Glm3,1 allotype according to the present invention can be constituted by the sequence of the constant region of the light chain of an IgG of animal origin, preferably of human origin, or be constituted by a hybrid sequence of different origins.

The IgG1 of Glm3,1 allotype according to the present invention can be a human, humanized or chimeric IgG1.

Within the meaning of the present invention, by “human IgG1” is meant an IgG1 in which the IgG1 has variable regions and constant parts of human origin.

Within the meaning of the present invention, by “chimeric IgG1” is meant an IgG1 in which the sequence of the light chain and/or of the heavy chain comprises or consists of a hybrid sequence originating from at least two distinct animals. In a non-limitative manner, the chimeric IgG1s can be in particular human/mouse or human/monkey hybrids.

Within the meaning of the present invention, by “humanized IgG1” is meant an IgG1 in which the IgG1 has hypervariable regions (CDRs) originating from an animal distinct from humans and framework regions and constant parts of human origin.

As the variable regions of the IgG1 are not involved in the recognition of the IgG1 by the FcRn protein, the present invention can be implemented with IgG1s in which the variable regions of the heavy chains and of the light chains recognize any known epitope. It can be a bispecific or immunoconjugated antibody.

In an embodiment, the invention relates to a method as defined above, in which the variable regions of the IgG1 of Glm3,1 allotype recognize an epitope selected from the group constituted by: TNF-α, CD52, PCSK9, mesothelin (MSLN), phosphatidylserine, CD125 (IL-5Rα), CD30, CanAg (glycoform of MUC-1), CCL2 (MCP-1), glypican 3 (GPC3), CD19, CD25 (IL-2Rα), CD319 (SLAMF7), CD115 (M-CSF receptor), DLL4 (delta-like ligand 4), MUC5AC (mucin 5AC), FOLR1 (folate receptor α chain), CD80, CD221 (IGF-1R), APP (amyloid precursor protein), carbonic anhydrase IX, IL-23 p19 subunit, EGF-R (HER-1, erbB1), CD51/CD61 (integrin α_{V}β₃), CD6, IgE, CD56, Her-3 (erbB3), CD194 (CCR4), GM-CSF, angiopoietin-2, CD20, CD248 (endosialin), oxidized LDLs, CD3ε, Nogo-A (reticulon 4), stx1 (shiga-like toxin 1), CD221 (IGF-1R), CD240D (Rhesus D antigen), CD126 (IL6-Rα), stx2 (shiga-like toxin 2, subunit A), IL-6, IL-23 p19 subunit, CD4, angiopoietin-2×VEGFCD27, integrin α₄β₇, CD70, EpCAM, vimentin, IL-13, CD274 (PD-L1), VEGF, IL-12/IL-23 chain p40, CD227 (MUC-1), CD40, EGFR×HER-3, CD11a, LFA-1 (integrin α_{L}β₂), CD266 (TWEAK), integrin 37, IgE, cMET (HGF-R), Egf17 (Epidermal Growth Factor-like domain 7), TNF-β, IL17A, HER-2 (erbB2), CD22, CD79b, IgE, CD309 (VEGFR2), IFN-α, CD304 (neuropilin 1 or NRP1), influenza virus haemagglutinin, Clostridium difficile toxin A, IFN-α/β/ω receptor chain 1, Toxin B, activin A receptor type IIB ActR-IIB, IL-1β, CD261 (TRAIL-R1), CD38, ganglioside GD2, influenza virus haemagglutinin, CXCL10 (IP-10), nectin 4, IL-9, TYRP1 (tyrosinase-related protein 1), EGCD308 (VEGFR1), MIF (Macrophage migration inhibitory factor), GM-CSF, CD261 (TRAIL-R1), CD74, respiratory syncytial virus F protein, CDw136 MST1R, CD135 (flt3), CD279 (PD-1), IL-17A, Staphylococcus aureus alpha toxin, EpCAM, rabies virus glycoprotein, HLA-DR, PD-L1, CD257 (BAFF), CD44, ganglioside GD3, CD18 (integrin β₂), IFN-γ, CD30, CD4, CD66e CEACAM5, CD33, CD23, IL-5, lipoteichoic acid, IL-4, CD4, Bacillus anthracis toxin PA, cytomegalovirus (CMV) glycoprotein B, FAP (fibroblast activation protein), CD2, CD227 (MUC-1), myostatin (GDF 8), Staphylococcus aureus clumping factor A, CD154, HBs (hepatitis B) antigen and stx2 (shiga-like toxin 2, subunit B).

In an embodiment, the invention relates to a method as defined above, in which the variable regions of the IgG1 of Glm3,1 allotype recognize an epitope selected from the group constituted by: TNF-α, PCSK9, mesothelin (MSLN), phosphatidylserine, CD125 (IL-5Rα), CD30, CanAg (glycoform of MUC-1), CCL2 (MCP-1), glypican 3 (GPC3), CD19, CD25 (IL-2Rα), CD319 (SLAMF7), CD115 (M-CSF receptor), DLL4 (delta-like ligand 4), MUC5AC (mucin 5AC), FOLR1 (folate receptor α chain), CD80, CD221 (IGF-1R), APP (amyloid precursor protein), carbonic anhydrase IX, IL-23 p19 subunit, EGF-R (HER-1, erbB1), CD51/CD61 (integrin α_{V}β₃), CD6, IgE, CD56, Her-3 (erbB3), CD194 (CCR4), GM-CSF, angiopoietin-2, CD20, CD248 (endosialin), oxidized LDLs, CD3ε, Nogo-A (reticulon 4), stx1 (shiga-like toxin 1), CD221 (IGF-1R), CD240D (Rhesus D antigen), CD126 (IL6-Rα), stx2 (shiga-like toxin 2, subunit A), IL-6, IL-23 p19 subunit, CD4, angiopoietin-2×VEGFCD27, integrin α₄β₇, CD70, EpCAM, vimentin, IL-13, CD274 (PD-L1), VEGF, IL-12/IL-23 chain p40, CD227 (MUC-1), CD40, EGFR×HER-3, CD11a, LFA-1 (integrin α_{L}β₂), CD266 (TWEAK), integrin β₇, IgE, cMET (HGF-R), Egf17 (Epidermal Growth Factor-like domain 7), TNF-β, IL17A, HER-2 (erbB2), CD22, CD79b, IgE, CD309 (VEGFR2), IFN-α, CD304 (neuropilin 1 or NRP1), influenza virus haemagglutinin, Clostridium difficile toxin A, IFN-α/β/ω receptor chain 1, Toxin B, activin A receptor type IIB ActR-IIB, IL-1β, CD261 (TRAIL-R1), CD38, ganglioside GD2, influenza virus haemagglutinin, CXCL10 (IP-10), nectin 4, IL-9, TYRP1 (tyrosinase-related protein 1), EGCD308 (VEGFR1), MIF (Macrophage migration inhibitory factor), GM-CSF, CD261 (TRAIL-R1), CD74, respiratory syncytial virus F protein, CDw136 MST1R, CD135 (flt3), CD279 (PD-1), IL-17A, Staphylococcus aureus alpha toxin, EpCAM, rabies virus glycoprotein, HLA-DR, PD-L1, CD257 (BAFF), CD44, ganglioside GD3, CD18 (integrin β₂), IFN-γ, CD30, CD4, CD66e CEACAM5, CD33, CD23, IL-5, lipoteichoic acid, IL-4, CD4, Bacillus anthracis toxin PA, cytomegalovirus (CMV) glycoprotein B, FAP (fibroblast activation protein), CD2, CD227 (MUC-1), myostatin (GDF 8), Staphylococcus aureus clumping factor A, CD154, HBs (hepatitis B) antigen and stx2 (shiga-like toxin 2, subunit B).

The therapeutic IgG1s currently on the market or in the clinical trial phase are of Glm17, Glm17,1 or Glm3 allotype. The half-life of these IgG1s can therefore be improved by replacing the constant region of the heavy chain of these IgG1s with the constant region of the heavy chain of Glm3,1 allotype.

In an embodiment, the invention relates to a method as defined above, in which the variable regions of the IgG1 of Glm3,1 allotype are identical to those of an IgG1 selected from the group constituted by: adalimumab, alemtuzumab, alirocumab, amatuximab, antumab ravtansine, bavituximab, benralizumab, brentuximab vedotin, cantuzumab ravtansine, carlumab, codrituzumab, coltuximab ravtansine, daclizumab, denintuzumab mafodotin, elotuzumab, emactuzumab, enoticumab, ensituximab, farletuzumab, galiximab, ganitumab, gantenerumab, girentuximab, golimumab, guselkumab, imgatuzumab, infliximab, intetumumab, itolizumab, ligelizumab, lorvotuzumab mertansine, lumretuzumab, mogamulizumab, namilumab, nesvacumab, obinutuzumab, ocaratuzumab, ontuxizumab, orticumab, otelixizumab, ozanezumab, pritoxaximab, rituximab, robatumumab, roledumab, sarilumab, setoxaximab, siltuximab, sirukumab, solanezumab, teplizumab, tildrakizumab, tocilizumab, tregalizumab, ublituximab, vanucizumab, varlilumab, vedolizumab, vorsetuzumab, vorsetuzumab mafodotin, adecatumumab, pritumumab, anrukinzumab, atezolizumab, bevacizumab, briakinumab, clivatuzumab, dacetuzumab, duligotuzumab, efalizumab, enavatuzumab, etrolizumab, omalizumab, onartuzumab, parsatuzumab, pateclizumab, perakizumab, pertuzumab, pinatuzumab vedotin, polatuzumab vedotin, quilizumab, ramucirumab, rontalizumab, sifalimumab, trastuzumab, trastuzumab emtansine, vesencumab, cixutumumab, actoxumab, aducanumab, anifrolumab, basiliximab, bezlotoxumab, bimagrumab, canakinumab, cetuximab, clazakizumab, conatumumab, dalotuzumab, daratumumab, dinutuximab, diridavumab, eldelumab, enfortumab vedotin, enokizumab, etaracizumab, ficlatuzumab, flanvotumab, futuximab, icrucumab, imalumab, lenzilumab, lexatumumab, lodelcizumab, lucatumumab, milatuzumab, milatuzumab-doxorubicin, motavizumab, narnatumab, necitumumab, olaratumab, palivizumab, patritumab, pidilizumab, secukinumab, tigatuzumab, tosatoxumab, tucotuzumab celmoleukin, veltuzumab, zatuximab, epratuzumab, zalutumumab, rafivirumab, apolizumab, avelumab, bapineuzumab, belimumab, bivatuzumab, cantuzumab mertansine, ecromeximab, erlizumab, felvizumab, fontolizumab, iratumumab, keliximab, labetuzumab, labetuzumab tetraxetan, lintuzumab, lumiliximab, mapatumumab, mepolizumab, morolimumab, ocrelizumab, ofatumumab, pagibaximab, pascolizumab, priliximab, raxibacumab, regavirumab, sibrotuzumab, siplizumab, sontuzumab, stamulumab, talizumab, tefibazumab, teneliximab, toralizumab, tuvirumab, urtoxazumab, and zanolimumab.

In an embodiment, the invention relates to a method as defined above, in which the variable regions of the IgG1 of Glm3,1 allotype are identical to those of an IgG1 selected from the group constituted by: adalimumab, alemtuzumab, alirocumab, amatuximab, antumab ravtansine, bavituximab, benralizumab, brentuximab vedotin, cantuzumab ravtansine, carlumab, codrituzumab, coltuximab ravtansine, daclizumab, denintuzumab mafodotin, elotuzumab, emactuzumab, enoticumab, ensituximab, farletuzumab, galiximab, ganitumab, gantenerumab, girentuximab, golimumab, guselkumab, imgatuzumab, infliximab, intetumumab, itolizumab, ligelizumab, lorvotuzumab mertansine, lumretuzumab, mogamulizumab, namilumab, nesvacumab, obinutuzumab, ocaratuzumab, ontuxizumab, orticumab, otelixizumab, ozanezumab, pritoxaximab, rituximab, robatumumab, roledumab, sarilumab, setoxaximab, siltuximab, sirukumab, solanezumab, teplizumab, tildrakizumab, tocilizumab, tregalizumab, ublituximab, vanucizumab, varlilumab, vedolizumab, vorsetuzumab, vorsetuzumab mafodotin, adecatumumab, pritumumab, anrukinzumab, atezolizumab, bevacizumab, briakinumab, clivatuzumab, dacetuzumab, duligotuzumab, efalizumab, enavatuzumab, etrolizumab, omalizumab, onartuzumab, parsatuzumab, pateclizumab, perakizumab, pertuzumab, pinatuzumab vedotin, polatuzumab vedotin, quilizumab, ramucirumab, rontalizumab, sifalimumab, trastuzumab, trastuzumab emtansine, vesencumab, cixutumumab, actoxumab, aducanumab, anifrolumab, basiliximab, bezlotoxumab, bimagrumab, canakinumab, cetuximab, clazakizumab, conatumumab, dalotuzumab, daratumumab, dinutuximab, diridavumab, eldelumab, enfortumab vedotin, enokizumab, etaracizumab, ficlatuzumab, flanvotumab, futuximab, icrucumab, imalumab, lenzilumab, lexatumumab, lodelcizumab, lucatumumab, milatuzumab, milatuzumab-doxorubicin, motavizumab, narnatumab, necitumumab, olaratumab, palivizumab, patritumab, pidilizumab, secukinumab, tigatuzumab, tosatoxumab, tucotuzumab celmoleukin, veltuzumab, zatuximab, epratuzumab, zalutumumab and rafivirumab.

In an embodiment, the invention relates to a method as defined above, in which the variable regions of the IgG1 of Glm3,1 allotype are identical to those of an IgG1 selected from the group constituted by: adalimumab, alemtuzumab, alirocumab, amatuximab, antumab ravtansine, bavituximab, benralizumab, brentuximab vedotin, cantuzumab ravtansine, carlumab, codrituzumab, coltuximab ravtansine, daclizumab, denintuzumab mafodotin, elotuzumab, emactuzumab, enoticumab, ensituximab, farletuzumab, galiximab, ganitumab, gantenerumab, girentuximab, golimumab, guselkumab, imgatuzumab, infliximab, intetumumab, itolizumab, ligelizumab, lorvotuzumab mertansine, lumretuzumab, mogamulizumab, namilumab, nesvacumab, obinutuzumab, ocaratuzumab, ontuxizumab, orticumab, otelixizumab, ozanezumab, pritoxaximab, rituximab, robatumumab, roledumab, sarilumab, setoxaximab, siltuximab, sirukumab, solanezumab, teplizumab, tildrakizumab, tocilizumab, tregalizumab, ublituximab, vanucizumab, varlilumab, vedolizumab, vorsetuzumab, vorsetuzumab mafodotin, adecatumumab, pritumumab, anrukinzumab, atezolizumab, bevacizumab, briakinumab, clivatuzumab, dacetuzumab, duligotuzumab, efalizumab, enavatuzumab, etrolizumab, omalizumab, onartuzumab, parsatuzumab, pateclizumab, perakizumab, pertuzumab, pinatuzumab vedotin, polatuzumab vedotin, quilizumab, ramucirumab, rontalizumab, sifalimumab, trastuzumab, trastuzumab emtansine, vesencumab, cixutumumab, actoxumab, aducanumab, anifrolumab, basiliximab, bezlotoxumab, bimagrumab, canakinumab, clazakizumab, conatumumab, dalotuzumab, daratumumab, dinutuximab, diridavumab, eldelumab, enfortumab vedotin, enokizumab, etaracizumab, ficlatuzumab, flanvotumab, futuximab, icrucumab, imalumab, lenzilumab, lexatumumab, lodelcizumab, lucatumumab, milatuzumab, milatuzumab-doxorubicin, motavizumab, narnatumab, necitumumab, olaratumab, palivizumab, patritumab, pidilizumab, secukinumab, tigatuzumab, tosatoxumab, tucotuzumab celmoleukin, veltuzumab, zatuximab, epratuzumab, zalutumumab and rafivirumab.

The IgG1 of Glm3,1 allotype obtained by the method can thus be a variant of Glm3,1 allotype of any known therapeutic IgG1.

A list of the therapeutic IgG1s and of the epitopes that they recognize is given in Table 3.

TABLE 3
List of IgG1 immunoglobulins and the respective epitopes thereof.

| | IgG1 (International Non- |
|---|---|
| Name of the target | proprietary Name (INN)) |
| TNF-α | adalimumab |
| CD52 | alemtuzumab |
| PCSK9 | alirocumab |
| mesothelin (MSLN) | amatuximab |
| mesothelin (MSLN) | antumab ravtansine |
| phosphatidylserine | bavituximab |
| CD125 (IL-5Rα) | benralizumab |
| CD30 | brentuximab vedotin |
| CanAg (glycoform of MUC-1) | cantuzumab ravtansine |
| CCL2 (MCP-1) | carlumab |
| glypican 3 (GPC3) | codrituzumab |
| CD19 | coltuximab ravtansine |
| CD25 (IL-2Rα) | daclizumab |
| CD19 | denintuzumab mafodotin |
| CD319 (SLAMF7) | elotuzumab |
| CD115 (M-CSFR) | emactuzumab |
| DLL4 (delta-like ligand 4) | enoticumab |
| MUC5AC (mucin 5AC) | ensituximab |
| FOLR1 (folate receptor α chain) | farletuzumab |
| CD80 | galiximab |
| CD221 (IGF-1R) | ganitumab |
| APP (amyloid precursor protein) | gantenerumab |
| carbonic anhydrase IX | girentuximab |
| TNF-α | golimumab |
| IL-23 p19 subunit | guselkumab |
| EGFR (erbB1, HER-1) | imgatuzumab |
| TNF-α | infliximab |
| CD51_CD61 (integrin α_{V}β₃) | intetumumab |
| CD6 | itolizumab |
| IgE | ligelizumab |
| CD56 | lorvotuzumab mertansine |
| Her-3 (erbB3 ) | lumretuzumab |
| CD194 (CCR4) | mogamulizumab |
| GM-CSF | namilumab |
| angiopoietin-2 | nesvacumab |
| CD20 | obinutuzumab |
| CD20 | ocaratuzumab |
| CD248 (endosialin) | ontuxizumab |
| oxidized LDLs | orticumab |
| CD3ε | otelixizumab |
| Nogo-A (reticulon 4) | ozanézumab |
| stx1 (shiga-like toxin 1) | pritoxaximab |
| CD20 | rituximab |
| CD221 (IGF-1R) | robatumumab |
| CD240D (Rhesus D antigen) | roledumab |
| CD126 (IL6-Rα) | sarilumab |
| stx2 (shiga-like toxin 2, subunit A) | setoxaximab |
| IL-6 | siltuximab |
| IL-6 | sirukumab |
| APP (amyloid precursor protein) | solanezumab |
| CD3ε | teplizumab |
| IL-23 p19 subunit | tildrakizumab |
| CD126 (IL6-Rα) | tocilizumab |
| CD4 | tregalizumab |
| CD20 | ublituximab |
| angiopoietin-2 × VEGF | vanucizumab |
| CD27 | varlilumab |
| integrin α₄β₇ | vedolizumab |
| CD70 | vorsetuzumab |
| CD70 | vorsetuzumab mafodotin |
| EpCAM | adecatumumab |
| vimentin | pritumumab |
| IL-13 | anrukinzumab |
| CD274 (PD-L1) | atezolizumab |
| VEGF | bevacizumab |
| IL-12/IL-23 chain p40 | briakinumab |
| CD227 (MUC-1) | clivatuzumab |
| CD40 | dacetuzumab |
| EGFR × HER-3 | duligotuzumab |
| CD11a LFA-1 (integrin α_{L}β₂) | efalizumab |
| CD266 (TWEAK) | enavatuzumab |
| integrin β₇ | etrolizumab |
| IgE | omalizumab |
| cMET (HGF-R) | onartuzumab |
| Egf17 (Epidermal Growth Factor-like | parsatuzumab |
| domain 7) | |
| TNF-β | pateclizumab |
| IL17A | perakizumab |
| HER-2 (erbB2) | pertuzumab |
| CD22 | pinatuzumab vedotin |
| CD79b | polatuzumab vedotin |
| IgE | quilizumab |
| CD309 (VEGFR2)) | ramucirumab |
| IFN-α | rontalizumab |
| IFN-α | sifalimumab |
| HER-2 (erbB2) | trastuzumab |
| HER-2 (erbB2) | trastuzumab emtansine |
| CD304 (neuropilin 1 orNRP1) | vesencumab |
| CD221 (IGF-1R) | cixutumumab |
| influenza virus haemagglutinin | firivumab |
| HER-2 (erbB2) | margetuximab |
| IL-12/IL-23 chain p40 | ustekinumab |
| Clostridium difficile toxin A | actoxumab |
| APP (amyloid precursor protein) | aducanumab |
| IFN-α/β/ω receptor chain 1 | anifrolumab |
| CD25 (IL-2Rα) | basiliximab |
| Toxin B | bezlotoxumab |
| activin A receptor type IIB ActR-IIB | bimagrumab |
| IL-1β | canakinumab |
| EGFR (erbB1, HER-1) | cetuximab |
| IL-6 | clazakizumab |
| CD261 (TRAIL-R1) | conatumumab |
| CD221 (IGF-1R) | dalotuzumab |
| CD38 | daratumumab |
| ganglioside GD2 | dinutuximab |
| influenza virus haemagglutinin | diridavumab |
| CXCL10 (IP-10) | eldelumab |
| nectin 4 | enfortumab vedotin |
| IL-9 | enokizumab |
| CD51_CD61 (integrin α_{V}β₃) | etaracizumab |
| cMET (HGF-R) | ficlatuzumab |
| TYRP1 (tyrosinase-related protein 1) | flanvotumab |
| EGFR (erbB1, HER-1) | futuximab |
| CD308 (VEGFR1) | icrucumab |
| MIF (Macrophage migration inhibitory factor) | imalumab |
| GM-CSF | lenzilumab |
| CD261 (TRAIL-R1) | lexatumumab |
| PCSK9 | lodelcizumab |
| CD40 | lucatumumab |
| CD74 | milatuzumab |
| CD74 | milatuzumab-doxorubicin |
| respiratory syncytial virus F protein | motavizumab |
| CDw136 MST1R | narnatumab |
| EGFR (erbB1, HER-1) | necitumumab |
| CD135 (flt3) | olaratumab |
| respiratory syncytial virus F protein | palivizumab |
| Her-3 (erbB3 ) | patritumab |
| CD279 (PD-1) | pidilizumab |
| IL-17A IL17A | secukinumab |
| CD261 (TRAIL-R1) | tigatuzumab |
| Staphylococcus aureus alpha toxin | tosatoxumab |
| EpCAM | tucotuzumab celmoleukin |
| CD20 | veltuzumab |
| EGFR (erbB1, HER-1) | zatuximab |
| CD22 | epratuzumab |
| EGFR (erbB1, HER-1) | zalutumumab |
| rabies virus glycoprotein | rafivirumab |
| HLA-DR | apolizumab |
| PD-L1, | avelumab |
| APP (amyloid precursor protein) | bapineuzumab |
| CD257 (BAFF) | belimumab |
| CD44 | bivatuzumab |
| CanAg (glycoform of MUC-1) | cantuzumab mertansine |
| ganglioside GD3 | ecromeximab |
| CD18 (integrin β₂) | erlizumab |
| respiratory syncytial virus F protein | felvizumab |
| IFN-γ | fontolizumab |
| CD30 | iratumumab |
| CD4 | keliximab |
| priliximab | labetuzumab |
| CD66e (CEACAM5) | labetuzumab tetraxetan |
| CD33 | lintuzumab |
| CD23 | lumiliximab |
| CD261 (TRAIL-R1) | mapatumumab |
| IL-5 | mepolizumab |
| CD240D (Rhesus D antigen) | morolimumab |
| CD20 | ocrelizumab |
| CD20 | ofatumumab |
| lipoteichoic acid | pagibaximab |
| IL-4 | pascolizumab |
| CD4 | priliximab |
| Bacillus anthracis toxin PA | raxibacumab |
| cytomegalovirus (CMV) glycoprotein B | regavirumab |
| FAP (fibroblast activation protein) | sibrotuzumab |
| CD2 | siplizumab |
| CD227 (MUC-1) | sontuzumab |
| myostatin (GDF 8) | stamulumab |
| IgE | talizumab |
| Staphylococcus aureus clumping factor A | tefibazumab |
| CD40 | teneliximab |
| CD154 | toralizumab |
| antigen HBs (hepatitis B) | tuvirumab |
| stx2 (shiga-like toxin 2, subunit B) | urtoxazumab |
| CD4 | zanolimumab |

In an embodiment, the invention relates to the method as defined above, in which the constant region of the heavy chain comprises sequence variations making it possible to improve the affinity of the IgG1 for the FcRn protein, in particular at the level of the CH2-CH3 constant regions of the heavy chain of the IgG1.

Such variations correspond to artificial mutations (deletion, insertion or substitution), resulting from human intervention.

In an embodiment, the invention relates to the method as defined above, in which the constant region of the heavy chain comprises sequence variations making it possible to modify (to increase or reduce) the binding of the IgG1 to C1q, FcγRI, FcγRIIA, FcγRIIB, FcγRIIIA and/or FcγRIIIB.

In an embodiment, the invention relates to the method as defined above, in which the constant region of the heavy chain comprises sequence variations making it possible to modify the potential T epitopes in order to reduce the immunogenicity.

In the invention, the sequence of the constant part of the heavy chain of the IgG1 of Glm3,1 allotype can thus comprise variations with respect to SEQ ID NO: 1, providing that the amino acids at positions 214, 356 and 358 are those corresponding to the Glm3,1 allotype.

These variations, making it possible to modify (to increase or to reduce) the binding of the IgG1 to C1q, FcγRI, FcγRIIA, FcγRIIB, FcγRIIIA and/or FcγRIIIB, can in particular be introduced in order to improve the recognition of the IgG1 by the immunity effector system. Advantageously, said variations can improve the affinity of the IgG1 for the FcRn protein.

Such variations are preferably introduced at the junction between the CH2-CH3 constant regions of the heavy chain, i.e. at the level of the site of recognition of the IgG1 by the FcRn protein, or into the CH2 (at the level of the small hinge).

Examples of variations making it possible to improve the affinity of an IgG1 for FcRn are for example described in U.S. Pat. No. 8,618,252. These variations can for example consist of at least one mutation at one of the following positions (EU nomenclature): 284, 285, 286, 288, 290, and 304, in particular one of the following mutations: a substitution at position 284 with a glutamate; a substitution at position 285 with a glutamate; a substitution at position 286 with an aspartate; a substitution at position 288 with a glutamate; a substitution at position 290 with a glutamate. Other mutations have also been described by Monnet et al., Front Immunol. 2015; 6: 39 (DOI: 10.3389/fimmu.2015.00039).

In another aspect, the invention relates to an IgG1 of Glm3,1 allotype for use thereof as a medicament, said IgG1 of Glm3,1 allotype not being selected from ustekinumab, firivumab and margetuximab.

In an embodiment, the invention relates to an IgG1 of Glm3,1 allotype for use thereof as a medicament, said IgG1 of Glm3,1 allotype not being selected from ustekinumab, firivumab, margetuximab and cetuximab.

In an embodiment, the invention relates to an IgG1 of Glm3,1 allotype for use thereof as a medicament, said IgG1 of Glm3,1 allotype not being selected from ustekinumab, firivumab, margetuximab and cetuximab and is not an IgG1 recognizing the WT1 (Wilm's Tumor Oncogene Protein) antigen.

As the IgG1 of Glm3,1 allotype has a better affinity for the FcRn receptor, the latter will have a better half-life duration compared with the IgG1s of Glm3, Glm17,1 and/or Glm17 allotype.

An IgG1 of Glm3,1 allotype with a greater half-life can consequently be administered in lower doses and/or at longer intervals compared with the IgG1s of Glm3, Glm17,1 and/or Glm17 allotype.

If necessary, said IgG1 of Glm3,1 allotype can also be administered concomitantly with a pharmaceutical substance capable of reducing a possible immunogenic reaction of the patient to this IgG1 (in particular the production of anti-IgG1 antibodies of Glm3,1 allotype). In non-limitative manner, such a substance can be for example methotrexate.

The IgG1s of Glm3,1 allotype can be used for treating any type of population.

In the case of patients producing IgG1s of Glm3 and/or Glm17,1 allotype, the IgG1 of Glm3,1 allotype will be more effectively recycled by the FcRn protein and will have a better half-life duration compared with the IgG1 endogens. An IgG1 of Glm3,1 allotype can thus be administered at lower doses and/or at longer intervals compared with an IgG1 of Glm3, Glm17,1 and/or Glm17 allotype having the same variable regions in order to achieve the same effectiveness.

In an embodiment, the invention relates to an IgG1 of Glm3,1 allotype as defined above for use thereof as a medicament, in a patient all or some of whose endogenous IgG1 s are of Glm3 and/or Glm17,1 allotypes.

In an embodiment, the invention relates to an IgG1 of Glm3,1 allotype as defined above for use thereof as a medicament, in a Glm3/Glm3 or Glm17,1/Glm17,1 homozygous patient or in a Glm3/Glm17,1 heterozygous patient.

In the case of patients producing IgG1s of Glm3,1 allotype, the endogenous IgG1s of the patient have a greater affinity for the FcRn protein and a better half-life compared with the therapeutic IgG1s of “non-Glm3,1” allotype. The therapeutic IgG1s of Glm17, Glm17,1 or Glm3 allotype are therefore less effectively recycled by the FcRn protein. The administration of IgG1 of Glm3,1 allotype to these patients makes it possible to improve recognition thereof by the FcRn protein, recycling thereof and, consequently, the half-life thereof.

In an embodiment, the invention relates to an IgG1 of Glm3,1 allotype as defined above for use thereof as a medicament, in a patient all or some of whose endogenous IgG1 s are of Glm3,1 allotype.

The patient can be Glm3,1/Glm3,1 homozygous or Glm3,1, heterozygous, the patient's second genotypic determinant being able to be any one of the other combinations of known allotypes of the human species (Glm3, or Glm17,1).

The Glm3,1 allotype is present in particular in the populations of Mongoloid origin. Unexpectedly, the IgG1s of Glm3,1 allotype are more effectively recycled and have a longer lifetime in patients of Mongoloid origin compared with the therapeutic IgG1s of Glm17, Glm17,1 or Glm3 allotypes.

The present invention therefore relates to an IgG1 of Glm3,1 allotype as defined above for use thereof as a medicament, in a Glm3,1/Glm3,1 homozygous patient or in a Glm3,1/Glm3 or Glm3,1/Glm17,1 heterozygous patient.

In an embodiment, the invention relates to an IgG1 of Glm3,1 allotype as defined above for use thereof as a medicament, in which the constant region of the heavy chain is constituted by the sequence SEQ ID NO: 1.

In an embodiment, the invention relates to an IgG1 of Glm3,1 allotype as defined above for use thereof as a medicament, in which the constant region of the heavy chain is constituted by a sequence having at least 90%, in particular 91%, 92%, 93%, 94%, 95%, 96%, 97%, 98%, 99% or 100%, identity with the sequence SEQ ID NO: 1.

In an embodiment, the invention relates to an IgG1 of Glm3,1 allotype as defined above for use thereof as a medicament, in which the peptide sequence of the constant region of the heavy chain comprises variations making it possible to improve the affinity of the IgG1 of Glm3,1 allotype for the FcRn protein, in particular at the level of the junction between the CH2-CH3 constant regions of the heavy chain of the IgG1 of Glm3,1 allotype.

In an embodiment, the invention relates to an IgG1 of Glm3,1 allotype as defined above for use thereof as a medicament, in which the variable regions recognize an epitope selected from the group constituted by: TNF-α, CD52, PCSK9, mesothelin (MSLN), phosphatidylserine, CD125 (IL-5Rα), CD30, CanAg (glycoform of MUC-1), CCL2 (MCP-1), glypican 3 (GPC3), CD19, CD25 (IL-2Rα), CD319 (SLAMF7), CD115 (M-CSF receptor), DLL4 (delta-like ligand 4), MUC5AC (mucin 5AC), FOLR1 (folate receptor α chain), CD80, CD221 (IGF-1R), APP (amyloid precursor protein), carbonic anhydrase IX, IL-23 p19 subunit, EGF-R (HER-1, erbB1), CD51/CD61 (integrin α_{V}β₃), CD6, IgE, CD56, Her-3 (erbB3), CD194 (CCR4), GM-CSF, angiopoietin-2, CD20, CD248 (endosialin), oxidized LDLs, CD3c, Nogo-A (reticulon 4), stx1 (shiga-like toxin 1), CD221 (IGF-1R), CD240D (Rhesus D antigen), CD126 (IL6-Rα), stx2 (shiga-like toxin 2, subunit A), IL-6, IL-23 p19 subunit, CD4, angiopoietin-2×VEGFCD27, integrin α₄β₇, CD70, EpCAM, vimentin, IL-13, CD274 (PD-L1), VEGF, IL-12/IL-23 chain p40, CD227 (MUC-1), CD40, EGFR×HER-3, CD11a, LFA-1 (integrin α_{L}β₂), CD266 (TWEAK), integrin β₇, IgE, cMET (HGF-R), Egf17 (Epidermal Growth Factor-like domain 7), TNF-β, IL17A, HER-2 (erbB2), CD22, CD79b, IgE, CD309 (VEGFR2), IFN-α, CD304 (neuropilin 1 or NRP1), influenza virus haemagglutinin, Clostridium difficile toxin A, IFN-α/β/ω receptor chain 1, Toxin B, activin A receptor type IIB ActR-IIB, IL-1β, CD261 (TRAIL-R1), CD38, ganglioside GD2, influenza virus haemagglutinin, CXCL10 (IP-10), nectin 4, IL-9, TYRP1 (tyrosinase-related protein 1), EGCD308 (VEGFR1), MIF (Macrophage migration inhibitory factor), GM-CSF, CD261 (TRAIL-R1), CD74, respiratory syncytial virus F protein, CDw136 MST1R, CD135 (flt3), CD279 (PD-1), IL-17A, Staphylococcus aureus alpha toxin, EpCAM, rabies virus glycoprotein, HLA-DR, PD-L1, CD257 (BAFF), CD44, ganglioside GD3, CD18 (integrin β₂), IFN-γ, CD30, CD4, CD66e CEACAM5, CD33, CD23, IL-5, lipoteichoic acid, IL-4, CD4, Bacillus anthracis toxin PA, (CMV) cytomegalovirus glycoprotein B, FAP (fibroblast activation protein), CD2, CD227 (MUC-1), myostatin (GDF 8), Staphylococcus aureus clumping factor A, CD154, antigen HBs (hepatitis B) and stx2 (shiga-like toxin 2, subunit B).

In an embodiment, the invention relates to an IgG1 of Glm3,1 allotype as defined above for use thereof as a medicament, in which the variable regions recognize an epitope selected from the group constituted by: TNF-α, PCSK9, mesothelin (MSLN), phosphatidylserine, CD125 (IL-5Rα), CD30, CanAg (glycoform of MUC-1), CCL2 (MCP-1), glypican 3 (GPC3), CD19, CD25 (IL-2Rα), CD319 (SLAMF7), CD115 (M-CSF receptor), DLL4 (delta-like ligand 4), MUC5AC (mucin 5AC), FOLR1 (folate receptor α chain), CD80, CD221 (IGF-1R), APP (amyloid precursor protein), carbonic anhydrase IX, IL-23 p19 subunit, EGF-R (HER-1, erbB1), CD51/CD61 (integrin α_{V}β₃), CD6, IgE, CD56, Her-3 (erbB3), CD194 (CCR4), GM-CSF, angiopoietin-2, CD20, CD248 (endosialin), oxidized LDLs, CD3c, Nogo-A (reticulon 4), stx1 (shiga-like toxin 1), CD221 (IGF-1R), CD240D (Rhesus D antigen), CD126 (IL6-Rα), stx2 (shiga-like toxin 2, subunit A), IL-6, IL-23 p19 subunit, CD4, angiopoietin-2×VEGFCD27, integrin α₄β₇, CD70, EpCAM, vimentin, IL-13, CD274 (PD-L1), VEGF, IL-12/IL-23 chain p40, CD227 (MUC-1), CD40, EGFR×HER-3, CD11a, LFA-1 (integrin α_{L}β₂), CD266 (TWEAK), integrin β₇, IgE, cMET (HGF-R), Egf17 (Epidermal Growth Factor-like domain 7), TNF-β, IL17A, HER-2 (erbB2), CD22, CD79b, IgE, CD309 (VEGFR2), IFN-α, CD304 (neuropilin 1 or NRP1), influenza virus haemagglutinin, Clostridium difficile toxin A, IFN-α/β/ω receptor chain 1, Toxin B, activin A receptor type IIB ActR-IIB, IL-1β, CD261 (TRAIL-R1), CD38, ganglioside GD2, influenza virus haemagglutinin, CXCL10 (IP-10), nectin 4, IL-9, TYRP1 (tyrosinase-related protein 1), EGCD308 (VEGFR1), MIF (Macrophage migration inhibitory factor), GM-CSF, CD261 (TRAIL-R1), CD74, respiratory syncytial virus F protein, CDw136 MST1R, CD135 (flt3), CD279 (PD-1), IL-17A, Staphylococcus aureus alpha toxin, EpCAM, rabies virus glycoprotein, HLA-DR, PD-L1, CD257 (BAFF), CD44, ganglioside GD3, CD18 (integrin β₂), IFN-γ, CD30, CD4, CD66e CEACAM5, CD33, CD23, IL-5, lipoteichoic acid, IL-4, CD4, Bacillus anthracis toxin PA, cytomegalovirus (CMV) glycoprotein B, FAP (fibroblast activation protein), CD2, CD227 (MUC-1), myostatin (GDF 8), Staphylococcus aureus clumping factor A, CD154, antigen HBs (hepatitis B) and stx2 (shiga-like toxin 2, subunit B).

In an embodiment, the invention relates to an IgG1 of Glm3,1 allotype as defined above for use thereof as a medicament in which the variable regions are identical to those of an IgG1 selected from the group constituted by: adalimumab, alemtuzumab, alirocumab, amatuximab, antumab ravtansine, bavituximab, benralizumab, brentuximab vedotin, cantuzumab ravtansine, carlumab, codrituzumab, coltuximab ravtansine, daclizumab, denintuzumab mafodotin, elotuzumab, emactuzumab, enoticumab, ensituximab, farletuzumab, galiximab, ganitumab, gantenerumab, girentuximab, golimumab, guselkumab, imgatuzumab, infliximab, intetumumab, itolizumab, ligelizumab, lorvotuzumab mertansine, lumretuzumab, mogamulizumab, namilumab, nesvacumab, obinutuzumab, ocaratuzumab, ontuxizumab, orticumab, otelixizumab, ozanezumab, pritoxaximab, rituximab, robatumumab, roledumab, sarilumab, setoxaximab, siltuximab, sirukumab, solanezumab, teplizumab, tildrakizumab, tocilizumab, tregalizumab, ublituximab, vanucizumab, varlilumab, vedolizumab, vorsetuzumab, vorsetuzumab mafodotin, adecatumumab, pritumumab, anrukinzumab, atezolizumab, bevacizumab, briakinumab, clivatuzumab, dacetuzumab, duligotuzumab, efalizumab, enavatuzumab, etrolizumab, omalizumab, onartuzumab, parsatuzumab, pateclizumab, perakizumab, pertuzumab, pinatuzumab vedotin, polatuzumab vedotin, quilizumab, ramucirumab, rontalizumab, sifalimumab, trastuzumab, trastuzumab emtansine, vesencumab, cixutumumab, actoxumab, aducanumab, anifrolumab, basiliximab, bezlotoxumab, bimagrumab, canakinumab, cetuximab, clazakizumab, conatumumab, dalotuzumab, daratumumab, dinutuximab, diridavumab, eldelumab, enfortumab vedotin, enokizumab, etaracizumab, ficlatuzumab, flanvotumab, futuximab, icrucumab, imalumab, lenzilumab, lexatumumab, lodelcizumab, lucatumumab, milatuzumab, milatuzumab-doxorubicin, motavizumab, narnatumab, necitumumab, olaratumab, palivizumab, patritumab, pidilizumab, secukinumab, tigatuzumab, tosatoxumab, tucotuzumab celmoleukin, veltuzumab, zatuximab, epratuzumab, zalutumumab, rafivirumab, apolizumab, avelumab, bapineuzumab, belimumab, bivatuzumab, cantuzumab mertansine, ecromeximab, erlizumab, felvizumab, fontolizumab, iratumumab, keliximab, labetuzumab, labetuzumab tetraxetan, lintuzumab, lumiliximab, mapatumumab, mepolizumab, morolimumab, ocrelizumab, ofatumumab, pagibaximab, pascolizumab, priliximab, raxibacumab, regavirumab, sibrotuzumab, siplizumab, sontuzumab, stamulumab, talizumab, tefibazumab, teneliximab, toralizumab, tuvirumab, urtoxazumab, and zanolimumab.

In an embodiment, the invention relates to an IgG1 of Glm3,1 allotype as defined above for use thereof as a medicament, in which the variable regions are identical to those of an IgG1 selected from the group constituted by: adalimumab, alemtuzumab, alirocumab, amatuximab, antumab ravtansine, bavituximab, benralizumab, brentuximab vedotin, cantuzumab ravtansine, carlumab, codrituzumab, coltuximab ravtansine, daclizumab, denintuzumab mafodotin, elotuzumab, emactuzumab, enoticumab, ensituximab, farletuzumab, galiximab, ganitumab, gantenerumab, girentuximab, golimumab, guselkumab, imgatuzumab, infliximab, intetumumab, itolizumab, ligelizumab, lorvotuzumab mertansine, lumretuzumab, mogamulizumab, namilumab, nesvacumab, obinutuzumab, ocaratuzumab, ontuxizumab, orticumab, otelixizumab, ozanezumab, pritoxaximab, rituximab, robatumumab, roledumab, sarilumab, setoxaximab, siltuximab, sirukumab, solanezumab, teplizumab, tildrakizumab, tocilizumab, tregalizumab, ublituximab, vanucizumab, varlilumab, vedolizumab, vorsetuzumab, vorsetuzumab mafodotin, adecatumumab, pritumumab, anrukinzumab, atezolizumab, bevacizumab, briakinumab, clivatuzumab, dacetuzumab, duligotuzumab, efalizumab, enavatuzumab, etrolizumab, omalizumab, onartuzumab, parsatuzumab, pateclizumab, perakizumab, pertuzumab, pinatuzumab vedotin, polatuzumab vedotin, quilizumab, ramucirumab, rontalizumab, sifalimumab, trastuzumab, trastuzumab emtansine, vesencumab, cixutumumab, actoxumab, aducanumab, anifrolumab, basiliximab, bezlotoxumab, bimagrumab, canakinumab, cetuximab, clazakizumab, conatumumab, dalotuzumab, daratumumab, dinutuximab, diridavumab, eldelumab, enfortumab vedotin, enokizumab, etaracizumab, ficlatuzumab, flanvotumab, futuximab, icrucumab, imalumab, lenzilumab, lexatumumab, lodelcizumab, lucatumumab, milatuzumab, milatuzumab-doxorubicin, motavizumab, narnatumab, necitumumab, olaratumab, palivizumab, patritumab, pidilizumab, secukinumab, tigatuzumab, tosatoxumab, tucotuzumab celmoleukin, veltuzumab, zatuximab, epratuzumab, zalutumumab and rafivirumab.

In an embodiment, the invention relates to an IgG1 of Glm3,1 allotype as defined above for use thereof as a medicament, in which the variable regions are identical to those of an IgG1 selected from the group constituted by: adalimumab, alemtuzumab, alirocumab, amatuximab, antumab ravtansine, bavituximab, benralizumab, brentuximab vedotin, cantuzumab ravtansine, carlumab, codrituzumab, coltuximab ravtansine, daclizumab, denintuzumab mafodotin, elotuzumab, emactuzumab, enoticumab, ensituximab, farletuzumab, galiximab, ganitumab, gantenerumab, girentuximab, golimumab, guselkumab, imgatuzumab, infliximab, intetumumab, itolizumab, ligelizumab, lorvotuzumab mertansine, lumretuzumab, mogamulizumab, namilumab, nesvacumab, obinutuzumab, ocaratuzumab, ontuxizumab, orticumab, otelixizumab, ozanezumab, pritoxaximab, rituximab, robatumumab, roledumab, sarilumab, setoxaximab, siltuximab, sirukumab, solanezumab, teplizumab, tildrakizumab, tocilizumab, tregalizumab, ublituximab, vanucizumab, varlilumab, vedolizumab, vorsetuzumab, vorsetuzumab mafodotin, adecatumumab, pritumumab, anrukinzumab, atezolizumab, bevacizumab, briakinumab, clivatuzumab, dacetuzumab, duligotuzumab, efalizumab, enavatuzumab, etrolizumab, omalizumab, onartuzumab, parsatuzumab, pateclizumab, perakizumab, pertuzumab, pinatuzumab vedotin, polatuzumab vedotin, quilizumab, ramucirumab, rontalizumab, sifalimumab, trastuzumab, trastuzumab emtansine, vesencumab, cixutumumab, actoxumab, aducanumab, anifrolumab, basiliximab, bezlotoxumab, bimagrumab, canakinumab, clazakizumab, conatumumab, dalotuzumab, daratumumab, dinutuximab, diridavumab, eldelumab, enfortumab vedotin, enokizumab, etaracizumab, ficlatuzumab, flanvotumab, futuximab, icrucumab, imalumab, lenzilumab, lexatumumab, lodelcizumab, lucatumumab, milatuzumab, milatuzumab-doxorubicin, motavizumab, narnatumab, necitumumab, olaratumab, palivizumab, patritumab, pidilizumab, secukinumab, tigatuzumab, tosatoxumab, tucotuzumab celmoleukin, veltuzumab, zatuximab, epratuzumab, zalutumumab and rafivirumab.

In an embodiment, the invention relates to an IgG1 of Glm3,1 allotype as defined above for use thereof as a medicament, in which the variable regions of said IgG1 of Glm3,1 allotype specifically recognize TNF-α (Tumor Necrosis Factor-α).

In an embodiment, the invention relates to an IgG1 of Glm3,1 allotype as defined above for use thereof as a medicament, in which the variable regions are identical to those of adalimumab.

In an embodiment, the present invention relates to a variant of adalimumab of Glm3,1 allotype for use thereof as a medicament, in particular in a patient whose endogenous IgG1 s are of Glm3,1 allotype.

In an embodiment, the present invention relates to a variant of adalimumab of Glm3,1 allotype for use thereof as a medicament, in particular in a patient whose endogenous IgG1 s are of Glm3 and/or Glm17,1 allotype.

In an embodiment, the invention relates to an IgG1 of Glm3,1 allotype as defined above for use thereof as a medicament, in which the variable regions are identical to those of rituximab.

In an embodiment, the present invention relates to a variant of rituximab of Glm3,1 allotype for use thereof as a medicament, in particular in a patient whose endogenous IgG1 s are of Glm3,1 allotype.

In an embodiment, the present invention relates to a variant of rituximab of Glm3,1 allotype for use thereof as a medicament, in particular in a patient whose endogenous IgG1 s are of Glm3 and/or Glm17,1 allotype.

In an embodiment, the invention relates to an IgG1 of Glm3,1 allotype as defined above for use thereof as a medicament, in which the variable regions are identical to those of trastuzumab.

In an embodiment, the present invention relates to a variant of trastuzumab of Glm3,1 allotype for use thereof as a medicament, in particular in a patient whose endogenous IgG1 s are of Glm3,1 allotype.

In an embodiment, the present invention relates to a variant of trastuzumab of Glm3,1 allotype for use thereof as a medicament, in particular in a patient whose endogenous IgG1 s are of Glm3 and/or Glm17,1 allotype.

In an embodiment, the invention relates to an IgG1 of Glm3,1 allotype as defined above for use thereof as a medicament, in which the variable regions are identical to those of cetuximab.

In an embodiment, the present invention relates to a variant of cetuximab of Glm3,1 allotype for use thereof as a medicament, in particular in a patient whose endogenous IgG1 s are of Glm3,1 allotype.

In an embodiment, the present invention relates to a variant of cetuximab of Glm3,1 allotype for use thereof as a medicament, in particular in a patient whose endogenous IgG1 s are of Glm3 and/or Glm17,1 allotype.

In an embodiment, the invention relates to an IgG1 of Glm3,1 allotype as defined above for use thereof as a medicament, in which the variable regions of said IgG1 of Glm3,1 allotype are not directed against CD52.

In an embodiment, the invention relates to an IgG1 of Glm3,1 allotype as defined above for use thereof as a medicament, in which said IgG1 of Glm3,1 allotype is not a variant of alemtuzumab of Glm3,1 allotype.

In a particular aspect, the invention also relates to a combination of at least two IgG1s, at least one of which is an IgG1 of Glm3,1 allotype, for use thereof as a medicament.

In another aspect, the invention relates to an IgG1 of Glm3,1 allotype, as defined above, for use thereof in the treatment of a disease belonging to the group constituted by cancerous conditions, autoimmune diseases, immune disorders, dysimmune conditions, infectious diseases, inflammatory diseases, degenerative diseases, metabolic diseases, vascular diseases, and coagulation anomalies.

In particular, the variable regions of the IgG1 of Glm3,1 allotype can be selected to recognize an epitope the recognition of which allows the treatment of cancerous conditions, or cancers. Said cancerous conditions belong in particular to the group constituted by bladder cancer, breast cancer, head and neck cancer, prostate cancer, colorectal cancer, gastric cancer, melanoma, in particular metastatic melanoma, breast cancer, ovarian cancer, cervical cancer, endometrial cancer, kidney cancer, small cell lung cancer, large cell lung cancer, pancreatic cancer, multiple myeloma, Hodgkin's and non-Hodgkin's lymphoma, systemic anaplastic large cell lymphoma, leukaemias, in particular acute lymphoblastic leukaemia, chronic lymphoid leukaemia and acute myeloblastic leukaemia, glioblastoma and neuroblastoma.

In particular, the variable regions of the IgG1 of Glm3,1 allotype can be selected to recognize an epitope the recognition of which allows the treatment of an autoimmune disease, an immune disorder, a dysimmune condition, an inflammatory disease, a degenerative disease, a metabolic disease, a vascular disease, or a coagulation anomaly belonging to the group constituted by macular degeneration, hypercholesterolaemia, prevention of thromboses in the case of angioplasty, autoimmune allergic rhinitis, graft rejection, graft-versus-host disease, asthma, multiple sclerosis, haemolytic anaemia, thrombotic thrombocytopaenic purpura, allergic dermatitis, anaphylactic reactions, Quincke's oedema, rheumatoid arthritis, idiopathic juvenile arthritis, ankylosing spondylitis, psoriatic rheumatism, vasculitis, systemic lupus erythematosus, Sjögren's syndrome, haemorrhagic rectocolitis, arteriosclerosis, osteoarthritis, osteoporosis, acute and chronic respiratory infections, Crohn's disease, psoriasis, reversal of dabigatran-induced anticoagulation, Castleman's disease, Muckle-Wells syndrome, cryopyrinopathies, haemophilia.

The variable regions of the IgG1 of Glm3,1 allotype can be selected to recognize an epitope the recognition of which allows the treatment of a viral, parasitic or bacterial infection, such as for example a respiratory syncytial virus infection.

In an embodiment, the invention relates to an IgG1 of Glm3,1 allotype as defined above, for use thereof in the treatment of an inflammatory disorder involving TNF-α, in particular rheumatoid arthritis, idiopathic juvenile arthritis, ankylosing spondylitis, Crohn's disease, haemorrhagic rectocolitis, psoriasis, psoriatic rheumatism, suppurative hidradenitis.

In a particular embodiment, the present invention relates to an IgG1 as described above, in which the variable regions of the IgG1 specifically recognize TNF-α, for use thereof in the treatment of an inflammatory disorder involving TNF-α, in particular rheumatoid arthritis, idiopathic juvenile arthritis or ankylosing spondylitis.

Advantageously, said IgG1 is a variant of adalimumab of Glm3,1 allotype.

More advantageously, the present invention relates to an IgG1 in which the variable regions of the IgG1 recognize TNF-α, preferably a variant of adalimumab of Glm3,1 allotype, for use thereof in the treatment of an inflammatory disorder involving TNF-α, in particular rheumatoid arthritis, idiopathic juvenile arthritis or ankylosing spondylitis in a patient whose endogenous IgG1s are of Glm3,1 allotype.

More advantageously, the present invention relates to an IgG1 in which the variable regions of the IgG1 recognize TNF-α, preferably a variant of adalimumab of Glm3,1 allotype, for use thereof in the treatment of an inflammatory disorder involving TNF-α, in particular rheumatoid arthritis, idiopathic juvenile arthritis or ankylosing spondylitis in a patient whose endogenous IgG1s are of Glm3 and/or Glm17,1 allotype.

In an embodiment, the invention relates to an IgG1 of Glm3,1 allotype as defined above, for use thereof in the treatment of a pathology treated with rituximab.

In a particular embodiment, the present invention relates to an IgG1 as described above, in which the variable regions of the IgG1 specifically recognize CD20.

Advantageously, said IgG1 is a variant of rituximab of Glm3,1 allotype.

In an embodiment, the invention relates to an IgG1 of Glm3,1 allotype as defined above, for use thereof in the treatment of a pathology treated with trastuzumab.

In a particular embodiment, the present invention relates to an IgG1 as described above, in which the variable regions of the IgG1 specifically recognize the HER-2 molecule.

Advantageously, said IgG1 is a variant of trastuzumab of Glm3,1 allotype.

In an embodiment, the invention relates to an IgG1 of Glm3,1 allotype as defined above, for use thereof in the treatment of a pathology treated with cetuximab.

In a particular embodiment, the present invention relates to an IgG1 as described above, in which the variable regions of the IgG1 specifically recognize the EGF or EGFR receptor.

Advantageously, said IgG1 is a variant of cetuximab of Glm3,1 allotype.

In an embodiment, the present invention relates to an IgG1 of Glm3,1 allotype as described above, with the exception of an IgG1 in which the variable regions of said IgG1 recognize CD52, for use thereof in the treatment of cancer, in particular of B-cell chronic lymphocytic leukaemia (B-CLL) or of prolymphocytic leukaemia, or of multiple sclerosis. In this embodiment, the present invention therefore does not relate to a variant of alemtuzumab (or CAMPATH-1H) of Glm3,1 allotype for use thereof in the treatment of multiple sclerosis or of cancer, in particular of B-cell chronic lymphocytic leukaemia (B-CLL) or of prolymphocytic leukaemia.

Given that it is possible to reduce the dose of IgG1 administered to the patient suffering from the pathology treated with said IgG1, this reduction in the dose can take the form of a reduction in the single dose administered and/or a reduction in the frequency between two consecutive administrations.

The present invention also relates to the use of a constant region of a heavy chain of IgG1 of Glm3,1 allotype, for reducing the single dose and/or the frequency of administration of an IgG1.

The present invention also relates to a method for reducing the single dose and/or the frequency of administration of an IgG1 administered for the treatment of a pathology, comprising the following steps:
1) selecting an IgG1 of Glm3, Glm17 or Glm17,1 allotype used in the treatment of the pathology,
2) replacing the constant region of said IgG1 of Glm3, Glm17 or Glm17,1 allotype with the constant region of a heavy chain of IgG1 of Glm3,1 allotype,
3) administering the IgG1 of Glm3,1 allotype, obtained in step (2), to a patient in need thereof, in particular to a patient with the Glm3,1 allotype.

In another aspect, the invention relates to a pharmaceutical composition comprising as active ingredient, an IgG1 of Glm3,1 allotype as defined above and a pharmaceutically acceptable carrier.

By “pharmaceutically acceptable carrier” is meant, within the meaning of the present invention, a non-toxic material compatible with the body of a patient.

The pharmaceutical composition of the invention can be administered by intravenous route, in particular by injection or by gradual infusion, by intramuscular route, by subcutaneous route, by local route by means of infiltrations, by mouth, or by respiratory or pulmonary route by means of aerosols.

The preparations for parenteral administration can include sterile aqueous or non-aqueous solutions, suspensions or emulsions. Examples of non-aqueous solvents are propylene glycol, polyethylene glycol, vegetable oils, or injectable organic esters such as ethyl oleate. Aqueous carriers comprise water, alcohol/water solutions, emulsions or suspensions.

In an embodiment, the invention relates to a pharmaceutical composition as defined above, in which in which the variable regions of the IgG1 of Glm3,1 allotype are identical to those of adalimumab, of rituximab, of trastuzumab or of cetuximab.

The following figures and examples will better illustrate the invention, without however limiting its scope.

### FIGURE LEGENDS

FIG. 1. Results of surface plasmon resonance (SPR) analysis of the binding of the allotype variants of adalimumab to FcRn (□: Glm3,1; Δ: Glm17; *: Glm3; ●: Glm17,1). In order to compare the binding kinetics, the responses (y-axis, expressed in arbitrary units, RU) were measured at three time intervals (x-axis, in seconds) after injection of the antibody into the system: 180 seconds (denoted Binding in the figure), 200 seconds (denoted Stability 1 in the figure) and 580 seconds (denoted Stability 2 in the figure).

FIG. 2. Results of surface plasmon resonance (SPR) analysis, taking the Glm17,1 variant of adalimumab as reference. The results are expressed in percentages in relation to the reference (considered as 100%). Left-hand bar (white background): 180 seconds (Binding in FIG. 1); central bar (black dots on white background): 200 seconds (Stability 1 in FIG. 1); right-hand bar (white dots on black background): 580 seconds (Stability 2 in FIG. 1).

FIG. 3. Results of analysis of the binding of the allotype variants of adalimumab by flow cytometry. The fluorescence intensities are measured for each adalimumab variant at concentrations of 0, 1, 10, 25, 50 and 100 μg/mL. The results are expressed as the inhibition of the binding of rituximab labelled with AF488 to the membrane FcRn in percent. Rituximab-AF488 alone is considered as 100% binding. The IgA variant of adalimumab constitutes the negative control.

FIG. 4. Genetic map of the vector pFUSE-CHIg-hG1.

FIG. 5. Genetic map of the vector pFUSE2-CLIg-hk.

FIG. 6. Diagram showing the production of an IgG1.

### EXAMPLES

### Example 1—Adalimumab

In order to avoid any participation of the variable domain of the therapeutic antibody or the influence of other parameters (such as for example the buffer or the system used for producing the antibodies), the 4 allotype forms, Glm3, Glm3,1, Glm17 and Glm17,1, of the same IgG1, adalimumab (a human anti-TNF-α therapeutic antibody), were constructed and tested. The constant region of the heavy chain is therefore different in the 4 allotype forms of adalimumab, whereas the variable region of the heavy chain and of the light chain, as well as the constant region of the light chain, are identical in the 4 allotype forms of adalimumab. These IgG1 s therefore differ only in the constant region of the heavy chain.

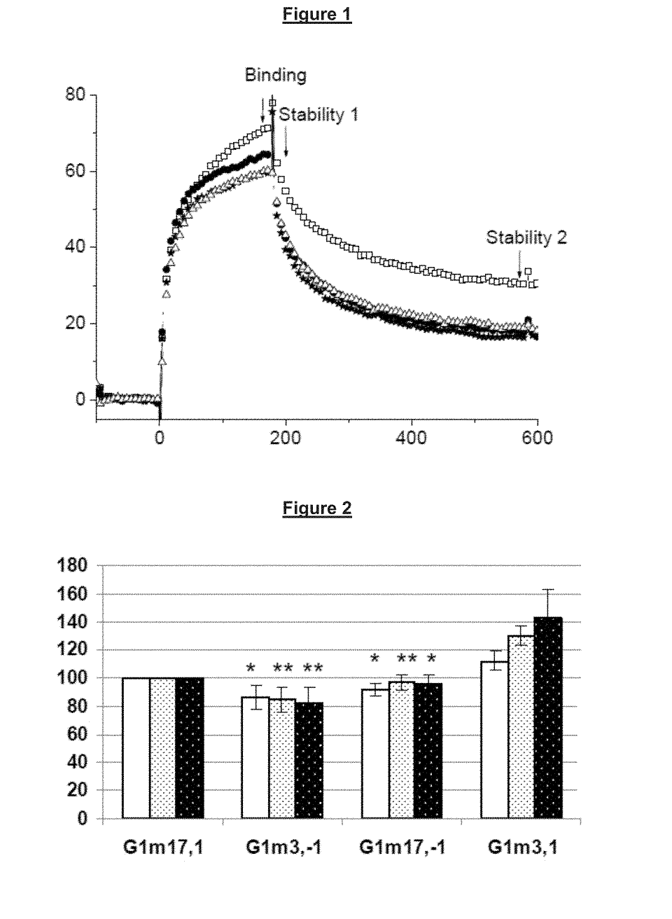

These IgG1s were first studied using SPR under the same conditions (FIGS. 1 and 2).

The values obtained with the Glm17,1 variant (such as commercial adalimumab) are considered as 100%.

These results indicate that the IgG1 of Glm3 allotype has a binding and a stability reduced by almost 15% with respect to the Glm17,1 allotype.

Unexpectedly, these results also indicate that adalimumab of Glm3,1 allotype proves to be the most effective in terms of binding affinity (+10%) and stability of the adalimumab/FcRn complex (up to +40%).

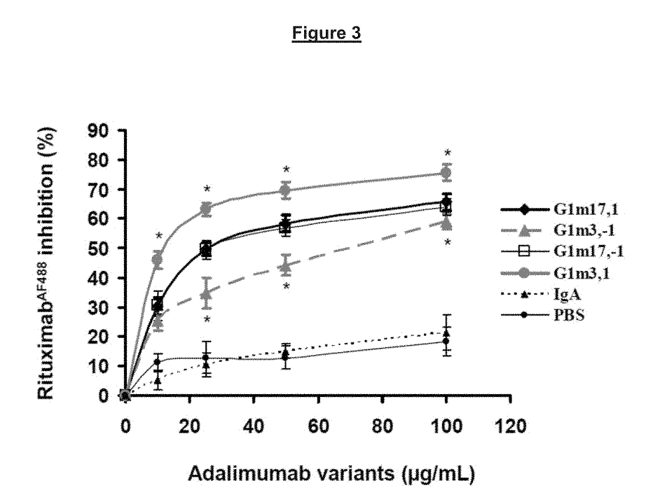

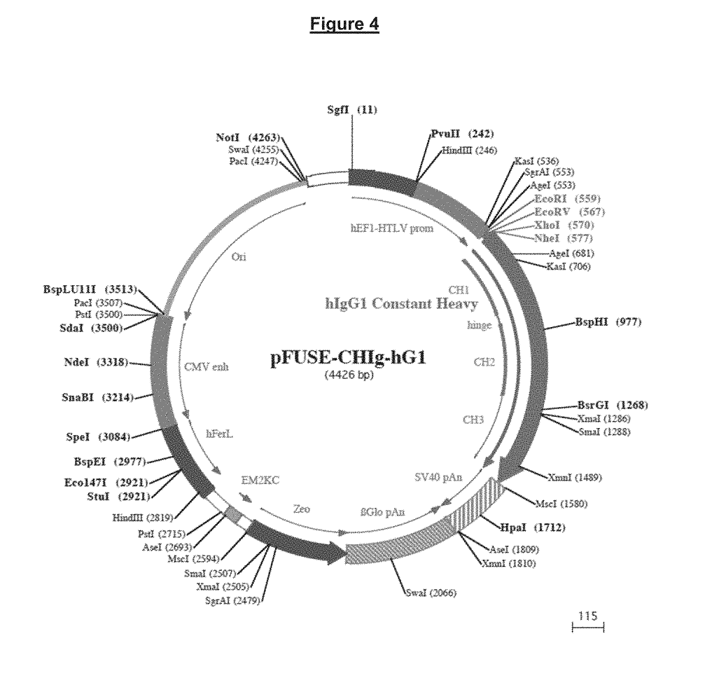

The results obtained with Jurkat_hFcRn cells reinforce these results and indicate that the variants of adalimumab of Glm3,1, Glm17,1 and Glm17 allotype have an ability to inhibit the binding of the rituximab-AF488 to FcRn at pH=6 that is greater than that of adalimumab of Glm3 allotype (FIG. 3).

Adalimumab of Glm3,1 allotype is, moreover, the most effective of the different allotypes.

These results show for the first time that variations of a residue in the CH1 domain in association with other residues in the CH3 domain are capable of modulating the affinity of an IgG1 for the FcRn protein, whereas these variations taken individually have no effect, and that they are not located in the zone of interaction with FcRn.

These results thus show that a therapeutic IgG1 of Glm3,1 allotype will have a greater half-life than an IgG1 that is identical but of a different allotype, irrespective of the allotype of the patient's endogenous IgG1s.

Taking into account the competition between a patient's endogenous IgG1s and the therapeutic IgG1s for the FcRn, these results indicate that the half-life of an IgG1 of Glm3, Glm17,1 or Glm17 allotype will be limited in a patient producing IgG1s of Glm3,1 allotype.

Conversely, therapeutic IgG1s of Glm3,1 allotype used in these same patients will have an improved half-life compared with the therapeutic IgG1s of Glm17,1, Glm17 or Glm3 allotype currently used in therapy.

This improved half-life can make it possible to reduce the single dose administered and/or the frequency of administration to the patients.

### Example 2—Trastuzumab

The 4 allotype forms, Glm3, Glm3,1, Glm17 and Glm17,1, of the same IgG1, trastuzumab (humanized anti-HER-2 therapeutic antibody), are constructed and tested. The constant region of the heavy chain is therefore different in the 4 allotype forms of trastuzumab, whereas the variable region of the heavy chain and of the light chain, as well as the constant region of the light chain, are identical in the 4 allotype forms of trastuzumab. These IgG1 s therefore differ only in the constant region of the heavy chain.

The IgG1 of Glm3,1 allotype is the most effective in terms of binding affinity and stability of the trastuzumab/FcRn complex.

### Example 3—Rituximab

The 4 allotype forms, Glm3, Glm3,1, Glm17 and Glm17,1, of the same IgG1, rituximab (chimeric anti-CD20 therapeutic antibody), are constructed and tested. The constant region of the heavy chain is therefore different in the 4 allotype forms of rituximab, whereas the variable region of the heavy chain and of the light chain, as well as the constant region of the light chain, are identical in the 4 allotype forms of rituximab. These IgG1 s therefore differ only in the constant region of the heavy chain.

The IgG1 of Glm3,1 allotype is the most effective in terms of binding affinity and stability of the rituximab/FcRn complex.

### Example 4—Cetuximab

The 4 allotype forms, Glm3, Glm3,1, Glm17 and Glm17,1, of the same IgG1, cetuximab (chimeric anti-EGFR therapeutic antibody), are constructed and tested. The constant region of the heavy chain is therefore different in the 4 allotype forms of cetuximab, whereas the variable region of the heavy chain and of the light chain, as well as the constant region of the light chain, are identical in the 4 allotype forms of cetuximab. These IgG1s therefore differ only in the constant region of the heavy chain.

The IgG1 of Glm3,1 allotype is the most effective in terms of binding affinity and stability of the cetuximab/FcRn complex.

Materials and Methods

Antibodies:

The antibodies used are adalimumab, rituximab, trastuzumab and cetuximab.

The allotype variants of adalimumab were produced from pFUSE-CHIg plasmids expressing the constant region of the heavy chain of the different human allotypes.

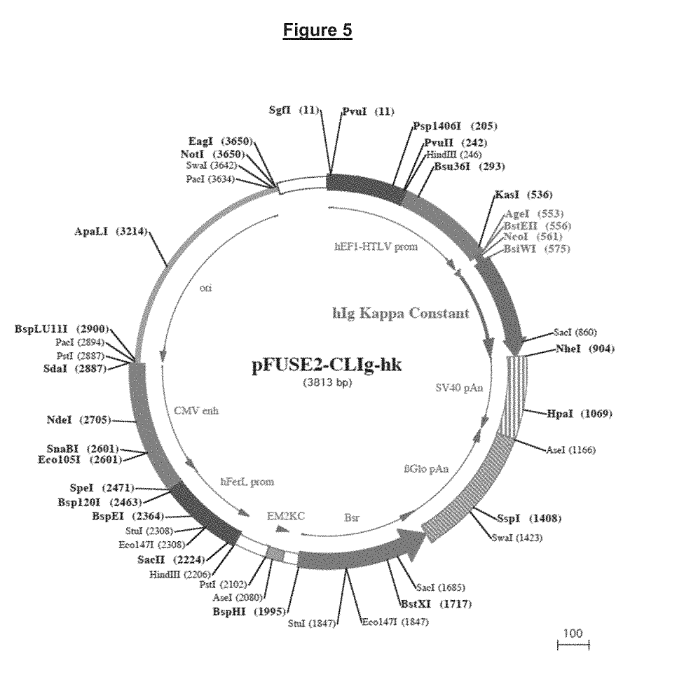

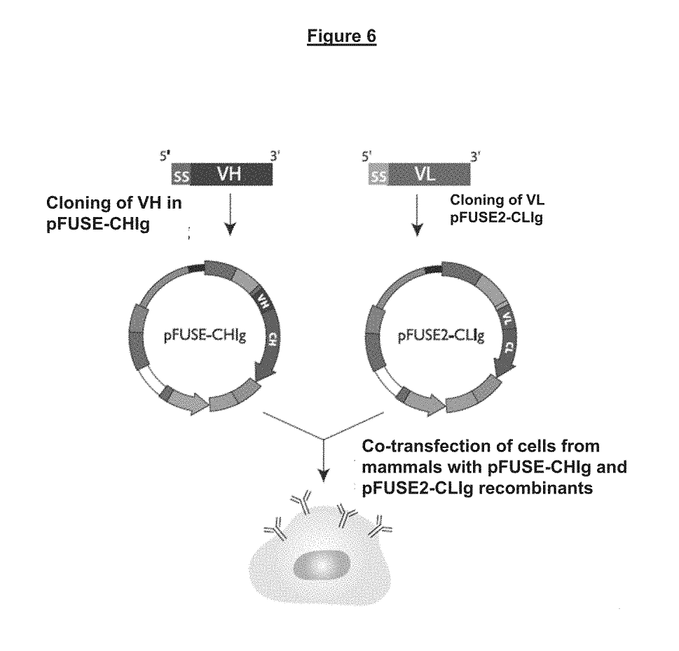

Each allotype variant of adalimumab was synthesized from the pFUSE-CHIg-hG1 (SEQ ID NO: 5) and pFUSE2-CLIg-hk (SEQ ID NO: 6) plasmids from InvivoGen. The sequence encoding the variable parts of the adalimumab was inserted into the cloning cassette (VH and VL) of both plasmids (FIGS. 4, 5 and 6). The sequence encoding the heavy chain of the pFUSE-CHIg was declined in each allotype. CHO (Chinese Hamster Ovary) cells were co-transfected with the two plasmids and the antibodies produced in the supernatant were purified by protein G affinity chromatography.

TABLE 4
Sequences of the vectors used
for constructing the allotype variants.

| Vector | Sequence |
|---|---|
| pFUSE-CHIg-hG1 | GGATCTGCGATCGCTCCGGTGCCCGTCAGTGGGCAGAGCGC |
| SEQ ID NO: 5 | ACATCGCCCACAGTCCCCGAGAAGTTGGGGGGAGGGGTCGG |
| The sequence of | CAATTGAACGGGTGCCTAGAGAAGGTGGCGCGGGGTAAACT |
| the constant region | GGGAAAGTGATGTCGTGTACTGGCTCCGCCTTTTTCCCGAGG |
| of the heavy chain | GTGGGGGAGAACCGTATATAAGTGCAGTAGTCGCCGTGAAC |
| is indicated in bold. | GTTCTTTTTCGCAACGGGTTTGCCGCCAGAACACAGCTGAA |
| | GCTTCGAGGGGCTCGCATCTCTCCTTCACGCGCCCGCCGCCC |
| | TACCTGAGGCCGCCATCCACGCCGGTTGAGTCGCGTTCTGCC |
| | GCCTCCCGCCTGTGGTGCCTCCTGAACTGCGTCCGCCGTCTA |
| | GGTAAGTTTAAAGCTCAGGTCGAGACCGGGCCTTTGTCCGG |
| | CGCTCCCTTGGAGCCTACCTAGACTCAGCCGGCTCTCCACGC |
| | TTTGCCTGACCCTGCTTGCTCAACTCTACGTCTTTGTTTCGTT |
| | TTCTGTTCTGCGCCGTTACAGATCCAAGCTGTGACCGGCGCC |
| | TACCTGAGATCACCGGTGAATTCGATATCTCGAGTGCTAGC |
| | ACCAAGGGCCCATCGGTCTTCCCCCTGGCACCCTCCTCC |
| | AAGAGCACCTCTGGGGGCACAGCGGCCCTGGGCTGCCT |
| | GGTCAAGGACTACTTCCCCGAACCGGTGACGGTGTCGTG |
| | GAACTCAGGCGCCCTGACCAGCGGCGTGCACACCTTCCC |
| | GGCTGTCCTACAGTCCTCAGGACTCTACTCCCTCAGCAG |
| | CGTGGTGACCGTGCCCTCCAGCAGCTTGGGCACCCAGA |
| | CCTACATCTGCAACGTGAATCACAAGCCCAGCAACACCA |
| | AGGTGGACAAGAAAGTTGAGCCCAAATCTTGTGACAAAA |
| | CTCACACATGCCCACCGTGCCCAGCACCTGAACTCCTGG |
| | GGGGACCGTCAGTCTTCCTCTTCCCCCCAAAACCCAAGG |
| | ACACCCTCATGATCTCCCGGACCCCTGAGGTCACATGCG |
| | TGGTGGTGGACGTGAGCCACGAAGACCCTGAGGTCAAG |
| | TTCAACTGGTACGTGGACGGCGTGGAGGTGCATAATGCC |
| | AAGACAAAGCCGCGGGAGGAGCAGTACAACAGCACGTA |
| | CCGTGTGGTCAGCGTCCTCACCGTCCTGCACCAGGACTG |
| | GCTGAATGGCAAGGAGTACAAGTGCAAGGTCTCCAACAA |
| | AGCCCTCCCAGCCCCCATCGAGAAAACCATCTCCAAAGC |
| | CAAAGGGCAGCCCCGAGAACCACAGGTGTACACCCTGC |
| | CCCCATCCCGGGAGGAGATGACCAAGAACCAGGTCAGC |
| | CTGACCTGCCTGGTCAAAGGCTTCTATCCCAGCGACATC |
| | GCCGTGGAGTGGGAGAGCAATGGGCAGCCGGAGAACAA |
| | CTACAAGACCACGCCTCCCGTGCTGGACTCCGACGGCTC |
| | CTTCTTCCTCTACAGCAAGCTCACCGTGGACAAGAGCAG |
| | GTGGCAGCAGGGGAACGTCTTCTCATGCTCCGTGATGCA |
| | TGAGGCTCTGCACAACCACTACACGCAGAAGAGCCTCTC |
| | CCTGTCTCCGGGTAAATGAGTCCTAGCTGGCCAGACATGA |
| | TAAGATACATTGATGAGTTTGGACAAACCACAACTAGAATG |
| | CAGTGAAAAAAATGCTTTATTTGTGAAATTTGTGATGCTATT |
| | GCTTTATTTGTAACCATTATAAGCTGCAATAAACAAGTTAAC |
| | AACAACAATTGCATTCATTTTATGTTTCAGGTTCAGGGGGAG |
| | GTGTGGGAGGTTTTTTAAAGCAAGTAAAACCTCTACAAATG |
| | TGGTATGGAATTAATTCTAAAATACAGCATAGCAAAACTTT |
| | AACCTCCAAATCAAGCCTCTACTTGAATCCTTTTCTGAGGGA |
| | TGAATAAGGCATAGGCATCAGGGGCTGTTGCCAATGTGCAT |
| | TAGCTGTTTGCAGCCTCACCTTCTTTCATGGAGTTTAAGATA |
| | TAGTGTATTTTCCCAAGGTTTGAACTAGCTCTTCATTTCTTTA |
| | TGTTTTAAATGCACTGACCTCCCACATTCCCTTTTTAGTAAA |
| | ATATTCAGAAATAATTTAAATACATCATTGCAATGAAAATA |
| | AATGTTTTTTATTAGGCAGAATCCAGATGCTCAAGGCCCTTC |
| | ATAATATCCCCCAGTTTAGTAGTTGGACTTAGGGAACAAAG |
| | GAACCTTTAATAGAAATTGGACAGCAAGAAAGCGAGCTTCT |
| | AGCTTATCCTCAGTCCTGCTCCTCTGCCACAAAGTGCACGCA |
| | GTTGCCGGCCGGGTCGCGCAGGGCGAACTCCCGCCCCCACG |
| | GCTGCTCGCCGATCTCGGTCATGGCCGGCCCGGAGGCGTCC |
| | CGGAAGTTCGTGGACACGACCTCCGACCACTCGGCGTACAG |
| | CTCGTCCAGGCCGCGCACCCACACCCAGGCCAGGGTGTTGT |
| | CCGGCACCACCTGGTCCTGGACCGCGCTGATGAACAGGGTC |
| | ACGTCGTCCCGGACCACACCGGCGAAGTCGTCCTCCACGAA |
| | GTCCCGGGAGAACCCGAGCCGGTCGGTCCAGAACTCGACCG |
| | CTCCGGCGACGTCGCGCGCGGTGAGCACCGGAACGGCACTG |
| | GTCAACTTGGCCATGATGGCTCCTCctgtcaggagaggaaa |
| | gagaagaaggttagtacaattgCTATAGTGAGTTGTATTAT |
| | ACTATGCAGATATACTATGCCAATGATTAATTGTCAAACTA |
| | GGGCTGCAgggttcatagtgccacttttcctgcactgcccc |
| | atctcctgcccaccctttcccaggcatagacagtcagtgac |
| | ttacCAAACTCACAGGAGGGAGAAGGCAGAAGCTTGAGACA |
| | GACCCGCGGGACCGCCGAACTGCGAGGGGACGTGGCTAGGGC |
| | GGCTTCTTTTATGGTGCGCCGGCCCTCGGAGGCAGGGCGCTC |
| | GGGGAGGCCTAGCGGCCAATCTGCGGTGGCAGGAGGCGGGGC |
| | CGAAGGCCGTGCCTGACCAATCCGGAGCACATAGGAGTCTC |
| | AGCCCCCCGCCCCAAAGCAAGGGGAAGTCACGCGCCTGTAG |
| | CGCCAGCGTGTTGTGAAATGGGGGCTTGGGGGGGTTGGGGC |
| | CCTGACTAGTCAAAACAAACTCCCATTGACGTCAATGGGGT |
| | GGAGACTTGGAAATCCCCGTGAGTCAAACCGCTATCCACGC |
| | CCATTGATGTACTGCCAAAACCGCATCATCATGGTAATAGC |
| | GATGACTAATACGTAGATGTACTGCCAAGTAGGAAAGTCCC |
| | ATAAGGTCATGTACTGGGCATAATGCCAGGCGGGCCATTTA |
| | CCGTCATTGACGTCAATAGGGGGCGTACTTGGCATATGATA |
| | CACTTGATGTACTGCCAAGTGGGCAGTTTACCGTAAATACTC |
| | CACCCATTGACGTCAATGGAAAGTCCCTATTGGCGTTACTAT |
| | GGGAACATACGTCATTATTGACGTCAATGGGCGGGGGTCGT |
| | TGGGCGGTCAGCCAGGCGGGCCATTTACCGTAAGTTATGTA |
| | ACGCCTGCAGGTTAATTAAGAACATGTGAGCAAAAGGCCAG |
| | CAAAAGGCCAGGAACCGTAAAAAGGCCGCGTTGCTGGCGTT |
| | TTTCCATAGGCTCCGCCCCCCTGACGAGCATCACAAAAATC |
| | GACGCTCAAGTCAGAGGTGGCGAAACCCGACAGGACTATA |
| | AAGATACCAGGCGTTTCCCCCTGGAAGCTCCCTCGTGCGCTC |
| | TCCTGTTCCGACCCTGCCGCTTACCGGATACCTGTCCGCCTT |
| | TCTCCCTTCGGGAAGCGTGGCGCTTTCTCATAGCTCACGCTG |
| | TAGGTATCTCAGTTCGGTGTAGGTCGTTCGCTCCAAGCTGGG |
| | CTGTGTGCACGAACCCCCCGTTCAGCCCGACCGCTGCGCCTT |
| | ATCCGGTAACTATCGTCTTGAGTCCAACCCGGTAAGACACG |
| | ACTTATCGCCACTGGCAGCAGCCACTGGTAACAGGATTAGC |
| | AGAGCGAGGTATGTAGGCGGTGCTACAGAGTTCTTGAAGTG |
| | GTGGCCTAACTACGGCTACACTAGAAGAACAGTATTTGGTA |
| | TCTGCGCTCTGCTGAAGCCAGTTACCTTCGGAAAAAGAGTT |
| | GGTAGCTCTTGATCCGGCAAACAAACCACCGCTGGTAGCGG |
| | TGGTTTTTTTGTTTGCAAGCAGCAGATTACGCGCAGAAAAA |
| | AAGGATCTCAAGAAGATCCTTTGATCTTTTCTACGGGGTCTG |
| | ACGCTCAGTGGAACGAAAACTCACGTTAAGGGATTTTGGTC |
| | ATGGCTAGTTAATTAACATTTAAATCAGCGGCCGCAATAAA |
| | ATATCTTTATTTTCATTACATCTGTGTGTTGGTTTTTTGTGTG |
| | AATCGTAACTAACATACGCTCTCCATCAAAACAAAACGAAA |
| | CAAAACAAACTAGCAAAATAGGCTGTCCCCAGTGCAAGTGC |
| | AGGTGCCAGAACATTTCTCTATCGAA |
| pFUSE2-CLIg-hk | GGATCTGCGATCGCTCCGGTGCCCGTCAGTGGGCAGAGCGC |
| SEQ ID NO: 6 | ACATCGCCCACAGTCCCCGAGAAGTTGGGGGGAGGGGTCGG |
| The sequence of | CAATTGAACGGGTGCCTAGAGAAGGTGGCGCGGGGTAAACT |
| the constant region | GGGAAAGTGATGTCGTGTACTGGCTCCGCCTTTTTCCCGAGG |
| of the kappa light | GTGGGGGAGAACCGTATATAAGTGCAGTAGTCGCCGTGAAC |
| chain is indicated | GTTCTTTTTCGCAACGGGTTTGCCGCCAGAACACAGCTGAA |
| in bold. | GCTTCGAGGGGCTCGCATCTCTCCTTCACGCGCCCGCCGCCC |
| | TACCTGAGGCCGCCATCCACGCCGGTTGAGTCGCGTTCTGCC |
| | GCCTCCCGCCTGTGGTGCCTCCTGAACTGCGTCCGCCGTCTA |
| | GGTAAGTTTAAAGCTCAGGTCGAGACCGGGCCTTTGTCCGG |
| | CGCTCCCTTGGAGCCTACCTAGACTCAGCCGGCTCTCCACGC |
| | TTTGCCTGACCCTGCTTGCTCAACTCTACGTCTTTGTTTCGTT |
| | TTCTGTTCTGCGCCGTTACAGATCCAAGCTGTGACCGGCGCC |
| | TACCTGAGATCACCGGTCACCATGGAAATCAAACGTACGGT |
| | GGCTGCACCATCTGTCTTCATCTTCCCGCCATCTGATGA |
| | GCAGTTGAAATCTGGAACTGCCTCTGTTGTGTGCCTGCT |
| | GAATAACTTCTATCCCAGAGAGGCCAAAGTACAGTGGAA |
| | GGTGGATAACGCCCTCCAATCGGGTAACTCCCAGGAGA |
| | GTGTCACAGAGCAGGACAGCAAGGACAGCACCTACAGC |
| | CTCAGCAGCACCCTGACGCTGAGCAAAGCAGACTACGA |
| | GAAACACAAAGTCTACGCCTGCGAAGTCACCCATCAGGG |
| | CCTGAGCTCGCCCGTCACAAAGAGCTTCAACAGGGGAG |
| | AGTGTTAGAGGGAGCTAGCTCGACATGATAAGATACATTG |
| | ATGAGTTTGGACAAACCACAACTAGAATGCAGTGAAAAAA |
| | ATGCTTTATTTGTGAAATTTGTGATGCTATTGCTTTATTTGTG |
| | AAATTTGTGATGCTATTGCTTTATTTGTAACCATTATAAGCT |
| | GCAATAAACAAGTTAACAACAACAATTGCATTCATTTTATG |
| | TTTCAGGTTCAGGGGGAGGTGTGGGAGGTTTTTTAAAGCAA |
| | GTAAAACCTCTACAAATGTGGTATGGAATTAATTCTAAAAT |
| | ACAGCATAGCAAAACTTTAACCTCCAAATCAAGCCTCTACT |
| | TGAATCCTTTTCTGAGGGATGAATAAGGCATAGGCATCAGG |
| | GGCTGTTGCCAATGTGCATTAGCTGTTTGCAGCCTCACCTTC |
| | TTTCATGGAGTTTAAGATATAGTGTATTTTCCCAAGGTTTGA |
| | ACTAGCTCTTCATTTCTTTATGTTTTAAATGCACTGACCTCCC |
| | ACATTCCCTTTTTAGTAAAATATTCAGAAATAATTTAAATAC |
| | ATCATTGCAATGAAAATAAATGTTTTTTATTAGGCAGAATCC |
| | AGATGCTCAAGGCCCTTCATAATATCCCCCAGTTTAGTAGTT |
| | GGACTTAGGGAACAAAGGAACCTTTAATAGAAATTGGACAG |
| | CAAGAAAGCGAGCTTCTAGCTTTAGTTCCTGGTGTACTTGAG |
| | GGGGATGAGTTCCTCAATGGTGGTTTTGACCAGCTTGCCATT |
| | CATCTCAATGAGCACAAAGCAGTCAGGAGCATAGTCAGAGA |
| | TGAGCTCTCTGCACATGCCACAGGGGCTGACCACCCTGATG |
| | GATCTGTCCACCTCATCAGAGTAGGGGTGCCTGACAGCCAC |
| | AATGGTGTCAAAGTCCTTCTGCCCGTTGCTCACAGCAGACCC |
| | AATGGCAATGGCTTCAGCACAGACAGTGACCCTGCCAATGT |
| | AGGCCTCAATGTGGACAGCAGAGATGATCTCCCCAGTCTTG |
| | GTCCTGATGGCCGCCCCGACATGGTGCTTGTTGTCCTCATAG |
| | AGCATGGTGATCTTCTCAGTGGCGACCTCCACCAGCTCCAG |
| | ATCCTGCTGAGAGATGTTGAAGGTCTTCATGATGGCTCCTCct |
| | gtcaggagaggaaagagaagaaggttagtacaattgCTATAG |
| | TGAGTTGTATTATACTATGCTTATGATTAATTGTCAAACTAG |
| | GGCTGCAgggttcatagtgccacttttcctgcactgccccatc |
| | tcctgcccaccctttcccaggcatagacagtcagtgacttac |
| | CAAACTCACAGGAGGGAGAAGGCAGAAGCTTGAGACAGAC |
| | CCGCGGGACCGCCGAACTGCGAGGGGACGTGGCTAGGGCG |
| | GCTTCTTTTATGGTGCGCCGGCCCTCGGAGGCAGGGCGCTC |
| | GGGGAGGCCTAGCGGCCAATCTGCGGTGGCAGGAGGCGGG |
| | GCCGAAGGCCGTGCCTGACCAATCCGGAGCACATAGGAGTC |
| | TCAGCCCCCCGCCCCAAAGCAAGGGGAAGTCACGCGCCTGT |
| | AGCGCCAGCGTGTTGTGAAATGGGGGCTTGGGGGGGTTGGG |
| | GCCCTGACTAGTCAAAACAAACTCCCATTGACGTCAATGGG |
| | GTGGAGACTTGGAAATCCCCGTGAGTCAAACCGCTATCCAC |
| | GCCCATTGATGTACTGCCAAAACCGCATCATCATGGTAATA |
| | GCGATGACTAATACGTAGATGTACTGCCAAGTAGGAAAGTC |
| | CCATAAGGTCATGTACTGGGCATAATGCCAGGCGGGCCATT |
| | TACCGTCATTGACGTCAATAGGGGGCGTACTTGGCATATGA |
| | TACACTTGATGTACTGCCAAGTGGGCAGTTTACCGTAAATA |
| | CTCCACCCATTGACGTCAATGGAAAGTCCCTATTGGCGTTAC |
| | TATGGGAACATACGTCATTATTGACGTCAATGGGCGGGGGT |
| | CGTTGGGCGGTCAGCCAGGCGGGCCATTTACCGTAAGTTAT |
| | GTAACGCCTGCAGGTTAATTAAGAACATGTGAGCAAAAGGC |
| | CAGCAAAAGGCCAGGAACCGTAAAAAGGCCGCGTTGCTGG |
| | CGTTTTTCCATAGGCTCCGCCCCCCTGACGAGCATCACAAAA |
| | ATCGACGCTCAAGTCAGAGGTGGCGAAACCCGACAGGACTA |
| | TAAAGATACCAGGCGTTTCCCCCTGGAAGCTCCCTCGTGCG |
| | CTCTCCTGTTCCGACCCTGCCGCTTACCGGATACCTGTCCGC |
| | CTTTCTCCCTTCGGGAAGCGTGGCGCTTTCTCATAGCTCACG |
| | CTGTAGGTATCTCAGTTCGGTGTAGGTCGTTCGCTCCAAGCT |
| | GGGCTGTGTGCACGAACCCCCCGTTCAGCCCGACCGCTGCG |
| | CCTTATCCGGTAACTATCGTCTTGAGTCCAACCCGGTAAGAC |
| | ACGACTTATCGCCACTGGCAGCAGCCACTGGTAACAGGATT |
| | AGCAGAGCGAGGTATGTAGGCGGTGCTACAGAGTTCTTGAA |
| | GTGGTGGCCTAACTACGGCTACACTAGAAGAACAGTATTTG |
| | GTATCTGCGCTCTGCTGAAGCCAGTTACCTTCGGAAAAAGA |
| | GTTGGTAGCTCTTGATCCGGCAAACAAACCACCGCTGGTAG |
| | CGGTGGTTTTTTTGTTTGCAAGCAGCAGATTACGCGCAGAA |
| | AAAAAGGATCTCAAGAAGATCCTTTGATCTTTTCTACGGGG |
| | TCTGACGCTCAGTGGAACGAAAACTCACGTTAAGGGATTTT |
| | GGTCATGGCTAGTTAATTAACATTTAAATCAGCGGCCGCAA |
| | TAAAATATCTTTATTTTCATTACATCTGTGTGTTGGTTTTTTG |
| | TGTGAATCGTAACTAACATACGCTCTCCATCAAAACAAAAC |
| | GAAACAAAACAAACTAGCAAAATAGGCTGTCCCCAGTGCA |
| | AGTGCAGGTGCCAGAACATTTCTCTATCGAA |

The allotype variants of rituximab, trastuzumab and cetuximab are produced using the same method.

Cell Lines:

The cell lines expressing or not expressing a truncated FcRn receptor are obtained by transfection of Jurkat cells with a plasmid containing the truncated human FcRn sequence, devoid of the 33 C-terminal amino acids of the protein.

The cells are maintained in an RPMI culture medium with 10% heat-inactivated fœtal calf serum, L-glutamine (2 mM), and G418 (1 mg/mL) added.

Measurement of Affinity by Flow Cytometry:

Rituximab is conjugated to the fluorescent label AF488 (rituximab-AF488) using the kit marketed by Invitrogen (Cergy-Pontoise, France) according to the manufacturer's instructions. Rituximab-AF488 is used at the concentration of 1 μg/mL in competition with either non-labelled rituximab or the different variants of adalimumab at a concentration of 1 to 100 times that of rituximab-AF488.

The Jurkat and Jurkat_ΔFcRn cell lines are mixed in a ratio of 1:1 in terms of number of cells. The cells (1×10⁵) are suspended in HBSS buffer adjusted to pH=6 with MES and incubated with rituximab-AF488 and the different antibodies at different concentrations.

After 30 minutes at 4° C., the fluorescence intensity is measured by flow cytometry (Coulter); the results are expressed as the inhibition of the binding of rituximab-AF488 to the JurkatΔhFcRn cells in percent.

The result with rituximab-AF488 alone is considered as corresponding to 100% binding.

Non-transfected Jurkat cells are used for assessing the non-specific binding of the antibodies.

Recombinant Human FcRn (hFcRn)

The sequence encoding human FcRn (cDNA clone MGC:1506 IMAGE:3163446) with its transmembrane and intra-cytoplasmic domains deleted is inserted into a pCMV6 Kan/Neo plasmid in order to obtain pCMV6hFcRn.

A histidine tag is then inserted into the plasmid resulting in pCMV6hFcRn-His.

The pCMV-SPORT6 plasmid encoding the human β2-microglobulin (NM_004048.2) was obtained from Origene.

The two plasmids (pCMV6hFcRn-His and pCMV-SPORT6 human β2-microglobulin) are transfected into HEK293 cells in order to produce soluble recombinant hFcRn.

The transfection system Large-scale transient transfection FreeStyle™ MAX Expression Systems (Invitrogen) is used according to the supplier's instructions in order to produce the soluble hFcRn-His.

The supernatants of the cell cultures containing the hFcRn-His fusion protein are collected, filtered and stored at −20° C.

The recombinant receptor hFcRn is purified with a Nickel-IMAC resin (HisPur Ni-NTA chromatography cartridge, Thermoscientific) and an AKTA purification instrument.

Surface Plasmon Resonance (SPR)

Real-time SPR analyses are carried out using a Biacore 3000 apparatus (GE Healthcare) at 25° C., at a flow rate of 50 μl/min in a 10 mM PBS buffer at pH=6 and 0.005% surfactant P20 (GE Healthcare). The human FcRn is immobilized by covalent binding on a CM5 biosensor chip (GE Healthcare) at a relatively low density (500 RU) by the EDC/NHS activation method, according to the supplier's instructions. The immunoglobulins are injected, over 180 seconds, at 50 nM into the biosensor where the FcRn has been immobilized and a control flow cell, the latter having been subjected to the same chemical treatment but being devoid of FcRn.

After a 400-second step of dissociation in the same buffer as previously, the surfaces of the sensor are regenerated with two pulses of PBS buffer at pH=7.4.

All the curves are assessed according to the double-reference method (Morton, T. A., and D. G. Myszka. 1998. Kinetic analysis of macromolecular interactions using surface plasmon resonance biosensors. Meth. Enzymol. 295: 268-294) using a two non-interacting binding sites model (BiaEvaluation 4.2) according to methods described previously (Vaughn, D. E., and P. J. Bjorkman. 1997, Biochemistry 36: 9374-9380; Martin, W. L., and P. J. Bjorkman. 1999, Biochemistry 38: 12639-12647; West, A. P., and P. J. Bjorkman. 2000, Biochemistry 39: 9698-9708; Datta-Mannan, A., D. R. Witcher, Y. Tang, J. Watkins, W. Jiang, V. J. and Wroblewski 2007, Drug Metab. Dispos. 35: 86-94).

Only the KD of the high-affinity site was retained for the correlation curve. In order to compare the binding kinetics of the different allotypes (50 nM) on the immobilized FcRn, the responses are measured in RU at three points on the curves: 180, 200 and 580 seconds, denoted binding, stability 1 and stability 2 respectively.

The data are read directly on the curve in order to eliminate any adaptation of the data. The ratio of the percentage is calculated taking the Glm17,1 allotype as reference (100%). All the curves are assessed by double reference. The stability of the coated surface is monitored by injection of rituximab (50 nM) as positive control, at the start and at the end of the series of experiments.

The experiments were carried out three times and replicated with freshly immobilized FcRn each time.

Statistical Analyses:

The data represent the average and the standard deviation of at least three experiments.

The Mann-Whitney test was used in order to determine the significant differences. The statistical analysis was carried out with GraphPad Prism 5 software.

Significance was defined at p≤0.05 and the level of significance is indicated in the figures as *p≤0.05, **p≤0.01 and ***p≤0.001.

1-14. (canceled)

15. Method for increasing the binding affinity of an IgG1 vis-à-vis the FcRn receptor, and/or increasing the stability of the complex formed by said IgG1 and FcRn, comprising a step in which the constant part of a heavy chain of an IgG1 of Glm3, Glm17 or Glm17,1 allotype is replaced with the constant part of a heavy chain of Glm3,1 allotype, in order to obtain an IgG1 of Glm3,1 allotype,
the binding affinity of the IgG1 of Glm3,1 allotype vis-à-vis the FcRn receptor being greater than the binding affinity of the IgG1 of Glm3, Glm17 or Glm17,1 allotype vis-à-vis the FcRn receptor, and/or
the stability of the complex formed by the IgG1 of Glm3,1 allotype and the FcRn, being greater than the stability of the complex formed by the IgG1 of Glm3, Glm17 or Glm17,1 allotype and the FcRn receptor.

16. Method according to claim 15, in which the binding affinity of the IgG1 of Glm3,1 allotype vis-à-vis the FcRn receptor, is at least 10% greater than the binding affinity of the IgG1 of Glm3, Glm17 or Glm17,1 allotype vis-à-vis the FcRn receptor.

17. Method according to claim 15, in which the stability of the complex formed by the IgG1 of Glm3,1 allotype and the FcRn, is at least 10% greater than the stability of the complex formed by the IgG1 of Glm3, Glm17 or Glm17,1 allotype and the FcRn receptor.

18. Method according to claim 15, in which the IgG1 of Glm3,1 allotype, has a half-life duration at least 10% greater than that of the IgG1 of Glm3, Glm17 or Glm17,1 allotype.

19. Method according to claim 15, in which:
a. the binding affinity of the IgG1 of Glm3,1 allotype vis-à-vis the FcRn receptor, is at least 10% greater than the binding affinity of the IgG1 of Glm3, Glm17 or Glm17,1 allotype vis-à-vis the FcRn receptor, and
b. the stability of the complex formed by the IgG1 of Glm3,1 allotype and the FcRn, is at least 10% greater than the stability of the complex formed by the IgG1 of Glm3, Glm17 or Glm17,1 allotype and the FcRn receptor.

20. Method according to claim 15, in which:
a. the binding affinity of the IgG1 of Glm3,1 allotype vis-à-vis the FcRn receptor, is at least 10% greater than the binding affinity of the IgG1 of Glm3, Glm17 or Glm17,1 allotype vis-à-vis the FcRn receptor, and
b. the IgG1 of Glm3,1 allotype, has a half-life duration at least 10% greater than that of the IgG1 of Glm3, Glm17 or Glm17,1 allotype.

21. Method according to claim 15, in which:
a. the stability of the complex formed by the IgG1 of Glm3,1 allotype and the FcRn, is at least 10% greater than the stability of the complex formed by the IgG1 of Glm3, Glm17 or Glm17,1 allotype and the FcRn receptor, and
b. the IgG1 of Glm3,1 allotype, has a half-life duration at least 10% greater than that of the IgG1 of Glm3, Glm17 or Glm17,1 allotype.

22. Method according to claim 15, in which:
a. the binding affinity of the IgG1 of Glm3,1 allotype vis-à-vis the FcRn receptor, is at least 10% greater than the binding affinity of the IgG1 of Glm3, Glm17 or Glm17,1 allotype vis-à-vis the FcRn receptor, and
b. the stability of the complex formed by the IgG1 of Glm3,1 allotype and the FcRn, is at least 10% greater than the stability of the complex formed by the IgG1 of Glm3, Glm17 or Glm17,1 allotype and the FcRn receptor, and
c. the IgG1 of Glm3,1 allotype, has a half-life duration at least 10% greater than that of the IgG1 of Glm3, Glm17 or Glm17,1 allotype.

23. Method according to claim 15, in which the variable regions of the IgG1 of Glm3,1 allotype recognize an epitope selected from the group constituted by: TNF-α, CD52, PCSK9, mesothelin (MSLN), phosphatidylserine, CD125 (IL-5Rα), CD30, CanAg (glycoform of MUC-1), CCL2 (MCP-1), glypican 3 (GPC3), CD19, CD25 (IL-2Rα), CD319 (SLAMF7), CD115 (M-CSF receptor), DLL4 (delta-like ligand 4), MUC5AC (mucin 5AC), FOLR1 (folate receptor α chain), CD80, CD221 (IGF-1R), APP (amyloid precursor protein), carbonic anhydrase IX, IL-23 p19 subunit, EGF-R (HER-1, erbB1), CD51/CD61 (integrin α_{V}β₃), CD6, IgE, CD56, Her-3 (erbB3), CD194 (CCR4), GM-CSF, angiopoietin-2, CD20, CD248 (endosialin), oxidized LDLs, CD3ε, Nogo-A (reticulon 4), stx1 (shiga-like toxin 1), CD221 (IGF-1R), CD240D (Rhesus D antigen), CD126 (IL6-Rα), stx2 (shiga-like toxin 2, subunit A), IL-6, IL-23 p19 subunit, CD4 angiopoietin-2×VEGFCD27, integrin α₄β₇, CD70, EpCAM, vimentin, IL-13, CD274 (PD-L1), VEGF, IL-12/IL-23 chain p40, CD227 (MUC-1), CD40, EGFR×HER-3, CD11a LFA-1 (integrin α_{L}β₂), CD266 (TWEAK), integrin β₇, IgE, cMET (HGF-R), Egf17 (Epidermal Growth Factor-like domain 7), TNF-β, IL17A, HER-2 (erbB2), CD22, CD79b, IgE, CD309 (VEGFR2), IFN-α, CD304 (neuropilin 1 or NRP1), influenza virus haemagglutinin, Clostridium difficile toxin A, IFN-α/β/ω receptor chain 1, Toxin B, activin A receptor type IIB ActR-IIB, IL-1β, CD261 (TRAIL-R1), CD38, ganglioside GD2, influenza virus haemagglutinin, CXCL10 (IP-10), nectin 4, IL-9, TYRP1 (tyrosinase-related protein 1) EGCD308 (VEGFR1), MIF (Macrophage migration inhibitory factor), GM-CSF, CD261 (TRAIL-R1), CD74, respiratory syncytial virus F protein, CDw136 MST1R, CD135 (flt3), CD279 (PD-1), IL-17A, Staphylococcus aureus alpha toxin, EpCAM, rabies virus glycoprotein, HLA-DR, PD-L1, CD257 (BAFF), CD44, ganglioside GD3, CD18 (integrin β₂), IFN-γ, CD30, CD4, CD66e CEACAM5, CD33, CD23, IL-5, lipoteichoic acid, IL-4, CD4, Bacillus anthracis toxin PA, cytomegalovirus (CMV) glycoprotein B, FAP (fibroblast activation protein), CD2, CD227 (MUC-1), myostatin (GDF 8), Staphylococcus aureus clumping factor A, CD154, antigen HBs (hepatitis B) and stx2 (shiga-like toxin 2, subunit B).

24. Method according to claim 15, in which the variable regions of the IgG1 of Glm3,1 allotype are identical to those of an IgG1 selected from the group constituted by: adalimumab, alemtuzumab, alirocumab, amatuximab, antumab ravtansine, bavituximab, benralizumab, brentuximab vedotin, cantuzumab ravtansine, carlumab, codrituzumab, coltuximab ravtansine, daclizumab, denintuzumab mafodotin, elotuzumab, emactuzumab, enoticumab, ensituximab, farletuzumab, galiximab, ganitumab, gantenerumab, girentuximab, golimumab, guselkumab, imgatuzumab, infliximab, intetumumab, itolizumab, ligelizumab, lorvotuzumab mertansine, lumretuzumab, mogamulizumab, namilumab, nesvacumab, obinutuzumab, ocaratuzumab, ontuxizumab, orticumab, otelixizumab, ozanezumab, pritoxaximab, rituximab, robatumumab, roledumab, sarilumab, setoxaximab, siltuximab, sirukumab, solanezumab, teplizumab, tildrakizumab, tocilizumab, tregalizumab, ublituximab, vanucizumab, varlilumab, vedolizumab, vorsetuzumab, vorsetuzumab mafodotin, adecatumumab, pritumumab, anrukinzumab, atezolizumab, bevacizumab, briakinumab, clivatuzumab, dacetuzumab, duligotuzumab, efalizumab, enavatuzumab, etrolizumab, omalizumab, onartuzumab, parsatuzumab, pateclizumab, perakizumab, pertuzumab, pinatuzumab vedotin, polatuzumab vedotin, quilizumab, ramucirumab, rontalizumab, sifalimumab, trastuzumab, trastuzumab emtansine), vesencumab, cixutumumab, actoxumab, aducanumab, anifrolumab, basiliximab, bezlotoxumab, bimagrumab, canakinumab, cetuximab, clazakizumab, conatumumab, dalotuzumab, daratumumab, dinutuximab, diridavumab, eldelumab, enfortumab vedotin, enokizumab, etaracizumab, ficlatuzumab, flanvotumab, futuximab, icrucumab, imalumab, lenzilumab, lexatumumab, lodelcizumab, lucatumumab, milatuzumab, milatuzumab-doxorubicin, motavizumab, narnatumab, necitumumab, olaratumab, palivizumab, patritumab, pidilizumab, secukinumab, tigatuzumab, tosatoxumab, tucotuzumab celmoleukin, veltuzumab, zatuximab, epratuzumab, zalutumumab, rafivirumab, apolizumab, avelumab, bapineuzumab, belimumab, bivatuzumab, cantuzumab mertansine, ecromeximab, erlizumab, felvizumab, fontolizumab, iratumumab, keliximab, labetuzumab, labetuzumab tetraxetan, lintuzumab, lumiliximab, mapatumumab, mepolizumab, morolimumab, ocrelizumab, ofatumumab, pagibaximab, pascolizumab, priliximab, raxibacumab, regavirumab, sibrotuzumab, siplizumab, sontuzumab, stamulumab, talizumab, tefibazumab, teneliximab, toralizumab, tuvirumab, urtoxazumab, and zanolimumab,
in particularly, an IgG1 selected from the group constituted by: adalimumab, rituximab, trastuzumab and cetuximab.

25. Method according to claim 15, in which the constant region of the heavy chain comprises sequence variations making it possible to improve the affinity of the IgG1 for the FcRn protein, in particular at the level of the junction between the CH2-CH3 constant regions of the heavy chain of the IgG1.

26. Method for treatment of a patient comprising the administration of IgG1 of Glm3,1 allotype, wherein IgG1 of Glm3,1 allotype is not selected from ustekinumab, firivumab, cetuximab and margetuximab and is not an IgG1 recognizing the antigen WT1.

27. Method according to claim 26, in a patient in whom all or some endogenous IgG1s are of Glm3 and/or Glm17,1 allotype.

28. Method according to claim 26, in a patient in whom all or some endogenous IgG1s are of Glm3,1 allotype.

29. Method according to claim 26, in which the variable regions of the IgG1 of Glm3,1 allotype recognize an epitope selected from the group constituted by: TNF-α, CD52, PCSK9, mesothelin (MSLN), phosphatidylserine, CD125 (IL-5Rα), CD30, CanAg (glycoform of MUC-1), CCL2 (MCP-1), glypican 3 (GPC3), CD19, CD25 (IL-2Rα), CD319 (SLAMF7), CD115 (M-CSF receptor), DLL4 (delta-like ligand 4), MUC5AC (mucin 5AC), FOLR1 (folate receptor α chain), CD80, CD221 (IGF-1R), APP (amyloid precursor protein), carbonic anhydrase IX, IL-23 p19 subunit, EGF-R (HER-1, erbB1), CD51/CD61 (integrin α_{V}β₃), CD6, IgE, CD56, Her-3 (erbB3), CD194 (CCR4), GM-CSF, angiopoietin-2, CD20, CD248 (endosialin), oxidized LDLs, CD3ε, Nogo-A (reticulon 4), stx1 (shiga-like toxin 1), CD221 (IGF-1R), CD240D (Rhesus D antigen), CD126 (IL6-Rα), stx2 (shiga-like toxin 2, subunit A), IL-6, IL-23 p19 subunit, CD4, angiopoietin-2×VEGFCD27, integrin α₄β₇, CD70, EpCAM, vimentin, IL-13, CD274 (PD-L1), VEGF, IL-12/IL-23 chain p40, CD227 (MUC-1), CD40, EGFR×HER-3, CD11a, LFA-1 (integrin α_{L}β₂), CD266 (TWEAK), integrin β₇, IgE, cMET (HGF-R), Egf17 (Epidermal Growth Factor-like domain 7), TNF-β, IL17A, HER-2 (erbB2), CD22, CD79b, IgE, CD309 (VEGFR2), IFN-α, CD304 (neuropilin 1 or NRP1), influenza virus haemagglutinin, Clostridium difficile toxin A, IFN-α/β/ω receptor chain 1, Toxin B, activin A receptor type IIB ActR-IIB, IL-13, CD261 (TRAIL-R1), CD38, ganglioside GD2, influenza virus haemagglutinin, CXCL10 (IP-10), nectin 4, IL-9, TYRP1 (tyrosinase-related protein 1), EGCD308 (VEGFR1), MIF (Macrophage migration inhibitory factor), GM-CSF, CD261 (TRAIL-R1), CD74, respiratory syncytial virus F protein, CDw136 MST1R, CD135 (flt3), CD279 (PD-1), IL-17A, Staphylococcus aureus alpha toxin, EpCAM, rabies virus glycoprotein, HLA-DR, PD-L1, CD257 (BAFF), CD44, ganglioside GD3, CD18 (integrin β₂), IFN-γ, CD30, CD4, CD66e, CEACAM5, CD33, CD23, IL-5, lipoteichoic acid, IL-4, CD4, Bacillus anthracis toxin PA, cytomegalovirus (CMV) glycoprotein B, FAP (fibroblast activation protein), CD2, CD227 (MUC-1), myostatin (GDF 8), Staphylococcus aureus clumping factor A, CD154, antigen HBs (hepatitis B) and stx2 (shiga-like toxin 2, subunit B).

30. Method according to claim 26, in which the variable regions of the IgG1 of Glm3,1 allotype are identical to those of an IgG1 selected from the group constituted by: adalimumab, alemtuzumab, alirocumab, amatuximab, antumab ravtansine, bavituximab, benralizumab, brentuximab vedotin, cantuzumab ravtansine, carlumab, codrituzumab, coltuximab ravtansine, daclizumab, denintuzumab mafodotin, elotuzumab, emactuzumab, enoticumab, ensituximab, farletuzumab, galiximab, ganitumab, gantenerumab, girentuximab, golimumab, guselkumab, imgatuzumab, infliximab, intetumumab, itolizumab, ligelizumab, lorvotuzumab mertansine, lumretuzumab, mogamulizumab, namilumab, nesvacumab, obinutuzumab, ocaratuzumab, ontuxizumab, orticumab, otelixizumab, ozanezumab, pritoxaximab, rituximab, robatumumab, roledumab, sarilumab, setoxaximab, siltuximab, sirukumab, solanezumab, teplizumab, tildrakizumab, tocilizumab, tregalizumab, ublituximab, vanucizumab, varlilumab, vedolizumab, vorsetuzumab, vorsetuzumab mafodotin, adecatumumab, pritumumab, anrukinzumab, atezolizumab, bevacizumab, briakinumab, clivatuzumab, dacetuzumab, duligotuzumab, efalizumab, enavatuzumab, etrolizumab, omalizumab, onartuzumab, parsatuzumab, pateclizumab, perakizumab, pertuzumab, pinatuzumab vedotin, polatuzumab vedotin, quilizumab, ramucirumab, rontalizumab, sifalimumab, trastuzumab, trastuzumab, vesencumab, cixutumumab, actoxumab, aducanumab, anifrolumab, basiliximab, bezlotoxumab, bimagrumab, canakinumab, cetuximab, clazakizumab, conatumumab, dalotuzumab, daratumumab, dinutuximab, diridavumab, eldelumab, enfortumab vedotin, enokizumab, etaracizumab, ficlatuzumab, flanvotumab, futuximab, icrucumab, imalumab, lenzilumab, lexatumumab, lodelcizumab, lucatumumab, milatuzumab, milatuzumab-doxorubicin, motavizumab, narnatumab, necitumumab, olaratumab, palivizumab, patritumab, pidilizumab, secukinumab, tigatuzumab, tosatoxumab, tucotuzumab celmoleukin, veltuzumab, zatuximab, epratuzumab, zalutumumab, rafivirumab, apolizumab, avelumab, bapineuzumab, belimumab, bivatuzumab, cantuzumab mertansine, ecromeximab, erlizumab, felvizumab, fontolizumab, iratumumab, keliximab, labetuzumab, labetuzumab tetraxetan, lintuzumab, lumiliximab, mapatumumab, mepolizumab, morolimumab, ocrelizumab, ofatumumab, pagibaximab, pascolizumab, priliximab, raxibacumab, regavirumab, sibrotuzumab, siplizumab, sontuzumab, stamulumab, talizumab, tefibazumab, teneliximab, toralizumab, tuvirumab, urtoxazumab, and zanolimumab,
in particularly, an IgG1 selected from the group constituted by: adalimumab, rituximab, trastuzumab and cetuximab.

31. Method for treatment according to claim 26 of a patient suffering from a disease belonging to the group constituted by cancerous conditions, autoimmune diseases, immune disorders, dysimmune disorders, infectious diseases, inflammatory diseases, degenerative diseases, metabolic diseases, vascular diseases, and coagulation anomalies, comprising the administration of IgG1 of Glm3,1 allotype.

32. Pharmaceutical composition comprising as active ingredient an IgG1 of Glm3,1 allotype as defined according to claim 26, and a pharmaceutically acceptable carrier.


SEQUENCE LISTING (6 sequences)

SEQ ID NO: 1 - protein, 330 aa, Artificial sequence
  Constant region of IgG1 G1m3,1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 330 aa, Artificial sequence
  Constant region of IgG1 G1m3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 330 aa, Artificial sequence
  Constant region of IgG1 G1m17
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 330 aa, Artificial sequence [sequence omitted]

SEQ ID NO: 5 - DNA, 4426 bp, Artificial sequence
  Vector
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggtgaattcgatatctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 6 - DNA, 3813 bp, Artificial sequence
  Vector
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggtcaccatggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa